(19) United States
(12) Patent Application Publication
(10) Pub. No.: US 2025/0043363 A1
(43) Pub. Date: Feb. 6, 2025

(54) METHODS AND SYSTEMS FOR ANALYZING AND UTILIZING CANCER TESTIS ANTIGEN BURDEN

(71) Applicant: OmniSeq, Inc., Buffalo, NY (US)

(72) Inventors: Sarabjot PABLA, Buffalo, NY (US); Robert John SEAGER, JR., Buffalo, NY (US); Erik VAN ROEY, Buffalo, NY (US); Shuang GAO, Buffalo, NY (US); Jeffrey M. CONROY, Williamsville, NY (US)

(73) Assignee: OmniSeq, Inc., Buffalo, NY (US)

(21) Appl. No.: 18/883,641

(22) Filed: Sep. 12, 2024

Related U.S. Application Data
(63) Continuation-in-part of application No. 18/055,292, filed on Nov. 14, 2022.
(60) Provisional application No. 63/263,913, filed on Nov. 11, 2021.

(51) Int. Cl. C12Q 1/6886; A61K 45/06; C07K 16/28

(52) U.S. Cl. CPC C12Q 1/6886; A61K 45/06; C07K 16/2818; C12Q 2600/106; C12Q 2600/158

(57) ABSTRACT

The present disclosure relates to methods for characterizing cancer testis antigen burden (“CTAB”), for predicting cancer survival outcomes using CTAB analysis, and for recommending and/or treating cancer using CTAB analysis. Particularly, aspects are directed to measuring expression of a panel of cancer testis antigen (CTA) gene markers in a tumor sample from a subject, determining a CTAB based on the measured expression of the CTA gene markers, classifying, by comparing to a reference CTAB score from the same cancer or tumor type, the CTAB score as a high-CTAB score or a low-CTAB score, and identifying the subject as responsive to a treatment wherein (i) the high-CTAB score is indicative of responsiveness to the treatment, or (ii) the low-CTAB score is indicative of responsiveness to the treatment.

### CROSS-REFERENCE TO RELATED APPLICATIONS

The present application is a Continuation-in-Part of U.S. application Ser. No. 18/055,292, filed Nov. 14, 2022, which claims priority and benefit from U.S. Provisional Application No. 63/263,913, filed on Nov. 11, 2021, the entire contents of which are incorporated herein by reference for all purposes.

### FIELD

The present disclosure is directed generally to diagnostic techniques for identifying subjects that respond to immune checkpoint inhibitor therapy based on their cancer testis antigen burden (CTAB) score and for recommending and/or treating cancer using CTAB score analysis.

### BACKGROUND

In the last two decades, immunotherapy has revolutionized cancer therapy. Immunotherapy or biological therapy is the treatment of disease by either activating or suppressing the immune system. Immunotherapies designed to elicit or amplify an immune response are classified as activation immunotherapies, while immunotherapies that reduce or suppress an immune response are classified as suppression immunotherapies. With respect to cancer, the goal of immunotherapy is to stimulate the immune system to destroy tumors. One way this is achieved is to use immune checkpoint inhibitors (ICIs) which target key regulators of the immune system that, when stimulated, can dampen the immune response to a stimulus. Thus, ICIs block inhibitory checkpoints and restore immune system function. In other words, they prevent the “off” signal from being sent, allowing immune cells (e.g., T cells) to kill cancer cells.

Because of this effect, ICIs have emerged as effective treatments in a variety of cancers, including breast cancers, colorectal cancers, and lung cancers like non-small cell lung cancer (NSCLC) which accounts for ˜50% of brain metastases. In addition, ICIs targeting the PD-1 receptor, and its ligands have proved highly effective in numerous cancer types. While the clinical utility of single agent ICI or in combination with chemotherapy has been well established, not all subjects benefit from ICI treatment and others develop resistance to these drugs over time. Much of the research in this area is geared towards finding ways to increase the efficacy of ICI drugs and to identify subjects more likely to respond to ICI treatment. Consequently, finding ways to increase the efficacy of these drugs and identify subjects who will derive the most benefit is an important theme of ongoing research. One important focus area in service of both goals is the identification of new targets for these drugs and biomarkers of subjects likely to respond to such treatments. Specifically for immuno-oncology, multiplex immunohistochemistry and digital special profiling of tumor biopsies have revealed complex tumor-immune interactions affecting response to checkpoint inhibitors. As a result, many immune-associated proteins are being explored as potential drug targets or biomarkers of treatment response. One such group of proteins are cancer testis antigens (CTAs).

CTAs are a category of tumor antigens that are normally expressed in the testes (specifically the male germ cells) and placenta but can also be aberrantly expressed in tumors of different histological origin. To date, approximately 250 proteins and associated genes are reported in literature. They are generally divided into two groups, CT-X antigens located on the X chromosome (52%) and non-X CTAs (48%). Most of these CTAs are expressed during spermatogenesis, but their function is still largely unknown. Epigenetic events, particularly DNA methylation and histone modifications, appear to be the primary mechanism regulating CTA restricted expression in adult somatic cells, as well as in cancer cells. Specifically, DNA hypomethylation and loss of histone modifications in the promoter regions of these genes are thought to be major contributors to their aberrant expression and ability to induce tumor-directed immune responses.

When expressed in cancer cells, CTAs are highly immunogenic and have the capacity to elicit cancer-specific immune responses in diverse malignancies. Evidence suggests that CTAs participate in tumorigenesis and progression by sustaining proliferative signaling, resisting cell death, evading growth suppressors, angiogenesis induction, major histocompatibility complex downregulation (immune evasion), deregulating cellular energetics, and genome instability. Moreover, the expression of CTAs appears to be correlated with tumor progression and are linked to cancer drug resistance. In addition, due to their restricted expression patterns and strong antigenicity, CTAs are also considered prognostic biomarkers and targets for immunotherapeutic interventions. Accordingly, CTAs have become a prime target of natural T cell response, immune cell-based therapies, cellular and antibody-based therapies, ICIs, immune checkpoint blockades (ICBs), and cancer vaccines research. In fact, multiple CTAs such as NY-ESO-1, MAGE-1, SSX2, and LAGE-1, are currently being investigated as potential targets and prospective biomarkers in various cancers.

### BRIEF SUMMARY

The present disclosure is directed to techniques for treating and providing robust CTA identification and quantitation. More specifically, techniques are disclosed herein for characterizing a subject's response to ICI treatment by robust CTA identification and quantitation. Co-expression of multiple CTA genes occurs in many tumor types and can be reliably detected using a targeted RNA-seq approach. Utilization of this co-expression pattern to calculate Cancer Testis Antigen Burden (CTAB) reveals tumor-type associated signatures, which can be associated with the overall survival (OS) of a subject. These immunogenic antigens expose the tumor cells to natural or immunotherapy augmented cell-based immune response, and thus CTAB can be used as a predictive marker for therapeutic response to ICIs.

Thus, provided herein is a method of treating a subject having cancer. The method includes obtaining sequencing reads from a biological sample from the subject, where the sequencing reads are generated from a sequencing assay. The sequencing reads are used to measure the expression of one or more genes for cancer testis antigens (CTAs) in a tumor sample from the subject having cancer. Based on the measured expression of the one or more CTA genes, a cancer testis antigen burden (CTAB) score is determined. The CTAB score is classified as a high-CTAB score or a low-CTAB score as compared to a reference CTAB score. Accordingly, the CTAB score is used to identify the subject as responsive to the treatment. In response to the CTAB score being a high-CTAB score, the subject is treated with the treatment. In response to the CTAB score being a low-CTAB score, the subject is treated with a different treatment. The method of treatment may further comprise obtaining slides of the biological sample. The biological sample may be a tumor tissue sample. For the slides, at least one slide is stained using hematoxylin and eosin staining, and the remaining slides are unstained. The stained slide(s) may be examined for pathological features associated with cancer to determine the severity of the cancer.

Also, provided herein is a method for identifying a response to a treatment in a subject having cancer. The method includes obtaining slides of a biological sample from the subject where at least one slide is stained and the remaining slides are unstained. The stained slide(s) are examined for pathological features associated with cancer to determine whether the subject is cancer positive, indeterminant, or cancer negative. In response to the subject being cancer positive or indeterminant, sequencing reads from the remaining unstained tissue sections are obtained by performing a sequencing assay. The sequencing reads are used to measure the expression of one or more genes for cancer testis antigens (CTAs) in a tumor sample from the subject having cancer. Based on the measured expression of the one or more CTA genes, a cancer testis antigen burden (CTAB) score is determined. The CTAB score is classified as a high-CTAB score or a low-CTAB score as compared to a reference CTAB score. Accordingly, the CTAB score is used to identify the subject as responsive to the treatment. The subject is identified as responsive to the treatment if they have a high-CTAB score. If the subject has a low-CTAB score, they are not identified as responsive to the treatment. Moreover, in response to the subject being cancer negative according to their pathological features, then the steps of obtaining sequencing reads and determining a CTAB score are not performed.

The method described herein further comprises treating the subject having cancer by administering an effective amount of the treatment when the CTAB score is indicative of responsiveness, or administering an effective amount of a different treatment when the CTAB score is not indicative of responsiveness. The treatment may be an immunotherapy alone or in combination with another anti-cancer therapy. The treatment can be an immune checkpoint inhibitor alone or in combination with another anti-cancer therapy. The immune checkpoint inhibitor can be selected from a group comprising pembrolizumab, ipilimumab, nivolumab, atezolizumab, cemiplimab, avelumab, or durvalumab. The method of treating the subject optionally includes repeating the method of identifying a response to a treatment at multiple time points to detect changes in the cancer and/or efficacy of treatment.

Also provided herein are methods directed towards identifying a subject having cancer who is responsive to a treatment based on their determined CTAB score and selecting the treatment for the subject according to their CTAB score. These methods include performing the method for identifying a response to a treatment in a subject having cancer and treating the subject with an effective amount of a treatment.

An assay system comprising components useful for the diagnosis and/or monitoring of subjects having cancer is also provided.

A kit comprising one or more reagents for performing the method of measuring the one or more genes for cancer testis antigens (CTAs) in a tumor sample from the subject having cancer is also provided.

The details of one or more embodiments are set forth in the description below. Other features, objects, and advantages will be apparent from the description and from the claims.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present invention will be better understood in view of the following non-limiting figures, in which:

FIG. 1 shows a representation of the 5250 samples from clinically tested FFPE tumors spanning 22 cancer types.

FIG. 2 is a heatmap representing cancer testis antigen (CTA) expression patterns across normal tissue sourced from the genotype-tissue expression database (GTEx). Data shows that robust expression of 14 CTA genes was only observed in testis tissue while immune-associated genes (CD8A and CD274) were expressed in most of the tissues.

FIG. 3 shows a flowchart showing calculation of normalized gene expression (GEX) rank from raw absolute read count values in the discovery cohort.

FIG. 4 shows a gene expression across all tumors for 17 CTAs classified as Positive (nRPM≥20) or Negative (nRPM<20).

FIGS. 5A and 5B show CTA co-expression in the discovery cohort. FIG. 5A is a correlation plot detailing the spearman correlations of 16 CTAs across the entire 5250 tumors of the discovery cohort. Of the 16 CTAs profiled, 13 were significantly co-expressed with one another while the expression of remaining 3 (MLANA, XAGE1B, and GAGE10) were largely independent from the others. As indicated by the black rectangles, 3 groups of CTAs were observed based on their expression. Negative values indicate a negative relationship between the compared CTA pair while a positive value indicates a positive relationship. All statistically significant (p≤0.05) correlations are denoted, and an “X” indicates a nonsignificant correlation. FIG. 5B is a network graph of CTA co-expression in the discovery cohort where the thickness and length of each line represent the absolute value of the correlation between two CTA (a thick, short line denotes a strong correlation and a thin, long line denotes a weak correlation). Only significant (p<0.5) correlations are shown.

FIGS. 6A and 6B show CTA co-expression data compiled from The Cancer Genome Atlas (TCGA) in order to confirm the CTA co-expression profile in the discovery cohort. FIG. 6A is a correlation plot detailing the pairwise Pearson correlations of 17 CTAs mRNA expression from 21,975 tumor samples. Of the 17 CTAs profiled, 15 were significantly co-expressed with one another while the expression of the remaining 2 (MILANA and GAGE10) were largely independent from the others. As indicated by the black rectangles, three groups of CTAs were observed based on their expression, which were similar to those observed in the discovery cohort. Negative values indicate a negative relationship between the compared CTA pair while a positive value indicates a positive relationship. All statistically significant (p≤0.05) correlations are denoted, and an “X” indicates a nonsignificant correlation. FIG. 6B is a network graph of CTA co-expression in the TCGA cohort where the thickness and length of each line represent the absolute value of the correlation between two CTA (a thick, short line denotes a strong correlation and a thin, long line denotes a weak correlation). Only significant (p<0.5) correlations are shown.

FIGS. 7A-7C show bar graphs of cancer testis antigen burden (CTAB) distributions in various cohorts. FIG. 7A shows the CTAB distribution in the discovery cohort having a median of 17, FIG. 7B shows the CTAB distribution in the TCGA cohort having a median of 247, and FIG. 7C shows the CTAB distribution in the retrospective cohort having a median of 193. The x- and y-axis represent gene expression ranks (i.e., percentile ranks) against a larger population.

FIGS. 8A-8C show box and whisker plots of CTAB distributions (ordered by median values) across the tumor types represented in each cohort. FIG. 8A shows the CTAB distribution for the 22 tumor types in the discovery cohort. FIG. 8B shows the CTAB distribution for the 22 tumor types in the TCGA cohort, and FIG. 8C shows the CTAB distribution in the retrospective cohort, which included only lung cancer patients. Overall, the median values for the same tumor types within the three cohorts were comparable.

FIGS. 9A and 9B show the distributions of CTAB for 22 tumor types in the discovery cohort and the TCGA cohorts. FIG. 9A shows that in the discovery cohort, the highest CTAB distribution was observed in melanoma suggesting this cancer has inherently higher CTA expression. The lowest CTAB scores were found in pancreatic, kidney and renal pelvis, and colorectal cancers. FIG. 9B shows that in the TCGA cohort, the highest CTAB distribution was observed in ovarian cancer and the lowest in prostate cancer.

FIG. 10A shows CoxPH regression analysis for CTAB, age, and gender effects in the TCGA cohort, while FIG. 10B shows CoxPH regression analysis for CTAB, age, and gender effects in the retrospective cohort.

FIG. 11A shows Kaplan-Meier survival analysis comparing CTAB positive (≥171) and negative (<171) groups for the TCGA cohort. FIG. 11B shows Kaplan-Meier survival analysis comparing CTAB positive (≥171) and negative (<171) groups for the retrospective cohort.

FIGS. 12A-12F are graphs showing Kaplan-Meier overall survival analyses for CTAB high and low groups across various histological and treatment subgroups in the retrospective cohort. For FIG. 12A, no significant difference in survival was observed between the high CTAB (bold line) and low CTAB (thin line) groups among all cases. For FIG. 12B, subjects treated with pembrolizumab monotherapy and with a high CTAB score demonstrated significantly better survival compared to those treated subjects with a low CTAB score. For FIG. 12C, subjects treated with a combination of pembrolizumab and chemotherapy did not show improved survival regardless of CTAB score. For FIG. 12D, when differences in treatment were not accounted for, the relationship between subjects with non-squamous NSCLC and CTAB was not significant. However, as shown in FIG. 12E, subjects with non-squamous NSCLC treated with pembrolizumab monotherapy and a high CTAB score did have significantly higher survival rates compared to those subjects with low CTAB. As shown in FIG. 12F, subjects with non-squamous NSCLC treated with pembrolizumab in combination with chemotherapy with lower CTAB scores trended towards having improved survival, however this was not found to be significant. Statistical comparison p-values indicated on each plot.

FIGS. 13A-13D are Kaplan-Meier (KM) overall survival graphs and response rate analyses comparing treatment groups within CTAB high and low groups in the retrospective cohort. Below each survival graph are the corresponding CTAB values for the high CTAB group (top) and the low CTAB group (bottom). FIG. 13A, for the CTAB high group, no significant difference in survival outcomes was observed between the two treatment groups. On the other hand, as shown in FIG. 13B, in the CTAB low group the combination therapy group exhibited significantly better survival than the pembrolizumab monotherapy group. FIG. 13C shows response rate analysis for subjects with high CTAB scores revealed that the pembrolizumab monotherapy group had a significantly greater proportion of subjects responding to treatment than the combination therapy group. FIG. 13D shows response rate analysis for subjects with low CTAB revealed that nearly 50% of subjects who received a subsequent treatment of pembrolizumab responded to the treatment compared to approximately 30% of combination therapy subjects. Fisher's Exact Test p-values indicated comparing the proportions of responders between treatment groups.

FIGS. 14A-14B show stacked bar graphs for the comparison of the proportion of subjects exhibiting stable disease (SD) in the pembrolizumab monotherapy and pembrolizumab-chemotherapy combination therapy groups for subjects in the retrospective cohort. For FIG. 14A, compared to the combination therapy group, subjects with a high CTAB score who received pembrolizumab treatment were significantly less likely to exhibit stable disease signs and instead displayed either progressive disease (PD), partial response (PR), or complete response (CR) (e.g., responders). For FIG. 14B, in the low CTAB group, the pembrolizumab monotherapy group showed a higher proportion of PD, PR, and CR responders compared to the combined therapy group; however, the SD distribution was similar in both treatment groups. The potential responses to treatment (i.e., stable disease (SD), progressive disease (PD), partial response (PR), or complete response (CR)) were based on the Response Evaluation Criteria in Solid Tumors (RECIST) v1.1 response grades. Fisher's Exact Test p-values indicated comparing the proportions of SD, PD, PR, CR.

FIGS. 15A and 15B show box plots illustrating the relationship between CTAB scores from the discovery cohort and established biomarkers. In FIG. 15A, the tumors were grouped based on their tumor proportion score (TPS), which indicates the percentage of tumor cells showing PD-L1 immuno-histochemistry (IHC) staining. No significant differences in CTAB were observed between the negative (TPS=0%), low (0% <TPS<50%), and high (TPS≥50%) subgroups, suggesting CTAB is an independent biomarker from PD-L1 expression. On the other hand, when the discovery cohort tumors were divided based on their tumor mutational burden (TMB) (FIG. 15B) a significance difference was observed between tumors having a TMB less than 10 mutations/Mega-base (mut/MB) and tumors with a TMB greater than 10 mutations/Mega-base. This suggests that as the number of mutations in the tumor increases, so does the CTAB score. Statistical comparisons between groups are indicated with Wilcoxon Rank-Sum p-values.

FIGS. 16A and 16B illustrate the correlation between CTAB with additional biomarkers, including PD-L1 IHC staining, PD-L1 gene expression, tumor immunogenic score (TIGS) and cell proliferation (CP), in the discovery cohort, while FIGS. 16C and 16D illustrate the same correlation in the cohort compiled from TCGA. FIGS. 16A and 16C are correlation plots showing Spearman correlation values, and FIGS. 16B and 16D show correlation network analysis between CTAB and the additional biomarkers. Overall, FIGS. 16A-16D show there were weak or nonsignificant correlations between CTAB and the additional biomarkers, regardless of cohort. This suggests that CTAB measures aspects of the tumor microenvironment independent from these biomarkers.

FIGS. 17A-17C show PCA and hierarchical clustering results combining five biomarkers of tumor and immune activity to classify tumors into groups based on overall tumor-immune dynamics. FIG. 17A shows an eigenvector plot detailing the influence of five constituent biomarkers on the first two principal components; FIG. 17B is a scatterplot detailing the classification of the 110 subject NSCLC validation cohort into four phenotypes (tumor dominant, proliferative, inflamed, and checkpoint); and FIG. 17C shows the distribution of the four phenotypes within the validation cohort.

FIGS. 18A-F shows Kaplan-Meier survival analyses for NSCLC validation cohort (n=110). FIG. 18A shows the survival analyses for NSCLC validation cohort stratified by PD-L1 IHC status. FIG. 18B shows the survival analyses for NSCLC validation cohort stratified by TMB status. FIG. 18C shows the survival analyses for NSCLC validation cohort stratified by cell proliferation. FIG. 18D shows the survival analyses for NSCLC validation cohort stratified by tumor immunogenic signature (TIGS). FIG. 18E shows the survival analyses for NSCLC validation cohort stratified by CTAB. and FIG. 18F shows the survival analyses for NSCLC validation cohort stratified by phenotype.

FIG. 19 shows CoxPH survival analysis of phenotype, the five constituent biomarkers, and sex and race as survival predictors.

FIG. 20 shows disease control rate (DCR) for the 110 NSCLC subject validation cohort as subdivided into the four phenotypes: tumor dominant; proliferative; inflamed; and checkpoint.

FIGS. 21A-21D show the results of the application of the phenotype classification to the retrospective cohort (Table 8) composed of 250 NSCLC patients treated with either the immune checkpoint inhibitor pembrolizumab alone or pembrolizumab and chemotherapy in combination. FIG. 21A is a treemap plot showing the proportions of the retrospective cohort classified into each of the four phenotypes. FIG. 21B is a boxplot showing the distributions of PD-L1 expression, as measured by immunohistochemistry (IHC) and expressed as a percent tumor proportion score (% TPS), observed in each of the phenotype groups in the retrospective cohort. In FIG. 21B, the brackets above the distributions represent statistically significant differences in PD-L1 expression between the phenotype groups, as determined by Wilcoxon Rank-Sum test, with p-values shown. FIG. 21C compares the overall response rate (ORR) observed in each of the phenotype groups in the retrospective cohort. Finally, FIG. 21D compares the ORR within the pembrolizumab and pembrolizumab+chemotherapy groups within each phenotype group in the retrospective cohort. In FIG. 21D, brackets above the bars represent statistically significant differences in ORR between the groups indicated by each bar, as determined by Fisher's Exact Test, with p-values shown.

FIGS. 22A-D show Kaplan-Meier survival analyses of the effects of treatment regimen on survival within the retrospective cohort. Each subplot compares patients treated with pembrolizumab alone to those treated with pembrolizumab and chemotherapy in combination (pembro+chemo) to determine whether a statistical association exists between treatment and survival.÷FIG. 22A shows 1 year survival for patients classified into the proliferative phenotype; FIG. 22B shows overall survival for patients classified into the proliferative phenotype; FIG. 22C shows 1 year survival for patients classified into the inflamed phenotype; and FIG. 22D shows overall survival for patients classified into the inflamed phenotype. P-values indicating the strength of the statistical association between treatment regimen and survival are indicated on each plot, median survival for each group is included in the legend above each plot, and risk tables are included below their respective plot.

### TERMS

As used herein, the terms “about,” “similarly,” “substantially,” and “approximately” and are defined as being largely but not necessarily wholly what is specified (and include wholly what is specified) as understood by one of ordinary skill in the art. In any disclosed embodiment, the term “about,” “similarly,” “substantially,” or “approximately” may be substituted with “within [a percentage] of” what is specified, where the percentage includes 0.1 percent, 1 percent, 5 percent, and 10 percent, etc.

As used herein, “administer,” “administering,” “administered” or “administration” refer to giving a dosage of a compound (e.g., an immunotherapy as described herein or a composition (e.g., a pharmaceutical composition, e.g., a pharmaceutical composition including an immunotherapy) to a subject. The compounds and/or compositions utilized in the methods described herein can be administered, for example, orally, intramuscularly, intravenously (e.g., by intravenous infusion), subcutaneously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, topically, locally, by inhalation, by injection, by infusion, by continuous infusion, by localized perfusion bathing target cells directly, by catheter, by lavage, in creams, or in lipid compositions. The method of administration can vary depending on various factors (e.g., the compound or composition being administered and the severity of the condition, disease, or disorder being treated).

As used herein, “anti-cancer therapy” refers to a therapy useful for treating a cancer. Examples of anti-cancer therapeutic agents include, but are not limited to, immunotherapies as described herein, growth inhibitory agents, cytotoxic agents, agents used in radiation therapy, anti-angiogenesis agents, apoptotic agents, anti-tubulin agents, and other agents to treat cancer, for example, anti-CD20 antibodies, platelet derived growth factor inhibitors, a COX-2 inhibitor, interferons, cytokines, antagonists (e.g., neutralizing antibodies), other bioactive and organic chemical agents, and the like. Combinations thereof are also included herein. An anti-cancer therapy as used herein can also be referred to a “non-immunotherapy” which in turn refers to any anti-cancer therapy excluding immunotherapies as defined herein.

As used herein, when an action is “based on” something, this means the action can be based at least in part on at least a part of the something.

As used herein, “biomarker” refers to an observable indicator, such as a predictive, diagnostic, and/or prognostic sign, that can be identified in a sample. The biomarker functions as a signal for a specific subtype of a disease or disorder (e.g., cancer), characterized by particular molecular, pathological, histological, and/or clinical features. In certain instances, a biomarker may manifest as a gene. Biomarkers encompass a variety of entities, including but not limited to polynucleotides (e.g., DNA and/or RNA), alterations in polynucleotide copy numbers (e.g., DNA copy numbers), polypeptides, modifications to polypeptides and polynucleotides (e.g., posttranslational modifications), carbohydrates, and/or glycolipid-based molecular markers.

As used herein, “biomarker signature,” “signature,” “biomarker expression signature,” or “expression signature” are used interchangeably herein and refer to one or a combination of biomarkers whose expression is an observable indicator, e.g., predictive, diagnostic, and/or prognostic. The biomarker signature may serve as an indicator of a particular subtype of a disease or disorder (e.g., cancer) characterized by certain molecular, pathological, histological, and/or clinical features. In some instances, the biomarker signature is a “gene signature.” The term “gene signature” is used interchangeably with “gene expression signature” and refers to one or a combination of polynucleotides whose expression is an indicator, e.g., predictive, diagnostic, and/or prognostic. In some embodiments, the biomarker signature is a “protein signature.” The term “protein signature” is used interchangeably with “protein expression signature” and refers to one or a combination of polypeptides whose expression is an indicator, e.g., predictive, diagnostic, and/or prognostic.

The biomarkers or biomarker signatures described herein with respect to CTAB are useful for identifying patient candidates for immunotherapy such as immune checkpoint therapies and/or predicting the outcome of immunotherapy in multiple cancer types, including without limitation, bladder cancer, lung cancer (e.g., non-small cell lung cancer), head and neck cancer, glioma, gliosarcoma, anaplastic astrocytoma, medulloblastoma, small cell lung carcinoma, cervical carcinoma, colon cancer, rectal cancer, chordoma, throat cancer, Kaposi's sarcoma, lymphangiosarcoma, lymphangioendotheliosarcoma, colorectal cancer, endometrium cancer, ovarian cancer, breast cancer, pancreatic cancer, prostate cancer, renal cell carcinoma, hepatic carcinoma, bile duct carcinoma, choriocarcinoma, seminoma, testicular tumor, Wilms' tumor, Ewing's tumor, bladder carcinoma, angiosarcoma, endotheliosarcoma, adenocarcinoma, sweat gland carcinoma, sebaceous gland sarcoma, papillary sarcoma, papillary adenosarcoma, cystadenosarcoma, bronchogenic carcinoma, medullary carcinoma, mastocytoma, mesotheliorma, synovioma, melanoma, leiomyosarcoma, rhabdomyosarcoma, neuroblastoma, retinoblastoma, oligodentroglioma, acoustic neuroma, hemangioblastoma, meningioma, pinealoma, ependymoma, craniopharyngioma, epithelial carcinoma, embryonal carcinoma, squamous cell carcinoma, base cell carcinoma, fibrosarcoma, myxoma, myxosarcoma, liposarcorna, chondrosarcoma, osteogenic sarcoma, and leukemia.

As used herein, “cancer” refers to an abnormal state or condition characterized by rapidly proliferating cell growth. Rapidly proliferating cells may be categorized as pathologic (i.e., characterizing or constituting a disease state), or may be categorized as non-pathologic (i.e., a deviation from normal but not associated with a disease state). In general, a cancer will be associated with the presence of one or more tumors (i.e., abnormal cell masses). The term “tumor” is meant to include all types of cancerous growths or oncogenic processes, metastatic tissues or malignantly transformed cells, tissues, or organs, irrespective of histopathologic type or stage of invasiveness. Examples of cancer include malignancies of various organ systems, such as lung cancers, breast cancers, thyroid cancers, lymphoid cancers, gastrointestinal cancers, and genito-urinary tract cancers. Cancer can also refer to adenocarcinomas, which include malignancies such as colon cancers, renal-cell carcinoma, prostate cancer and/or testicular tumors, non-small cell carcinoma of the lung, cancer of the small intestine, and cancer of the esophagus. Carcinomas are malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. An “adenocarcinoma” refers to a carcinoma derived from glandular tissue or in which the tumor cells form recognizable glandular structures. A “sarcoma” refers to a malignant tumor of mesenchymal derivation. “Melanoma” refers to a tumor arising from a melanocyte. Melanomas occur most commonly in the skin and are frequently observed to metastasize widely.

In certain instances, the cancer is lung cancer. As used herein, the term “lung cancer” refers to histologically or cytologically confirmed cancer of the lung. In some instances, the lung cancer is a carcinoma. In some instances, the lung cancer is an adenocarcinoma. In some instances, the lung cancer is a sarcoma. In some instances, the lung cancer is non-small cell lung cancer (NSCLC). In some instances, the lung cancer is NSCLC with non-squamous cell histology. In some instances, the NSCLC with squamous cell histology. In some instances, the lung cancer is a metastatic (i.e., spread from the lungs to other parts of the body) or a locally advanced lung cancer (i.e., spread from where it started in the lung to nearby tissue or lymph nodes, but not to other parts of the body). In some instances, the lung cancer is a recurrent lung cancer.

As used herein, the “expression level,” “amount,” or “level,” of a biomarker (e.g., a gene) is a detectable level in a biological sample. “Expression” generally refers to the process by which information (e.g., gene-encoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs). Expression levels can be measured by methods known to one skilled in the art and also disclosed herein. The expression level or amount of a biomarker can be used to identify/characterize a subject having a cancer who may be likely to respond to, or benefit from, a particular treatment (e.g., a treatment comprising immunotherapy). The expression level or amount of a biomarker provided herein in a subject having a cancer described herein can also be used to determine and/or track the benefit of an administered treatment over time.

As described herein, “likely”, “more likely”, or “highly likely” refers to a greater than 50% chance (e.g., 51%, 55%, 60%, 70%, 80%, 90%, and 100%). Furthermore, the term “significant” or “significantly” refers to a statistically significant result that has been predicted as unlikely to have occurred by chance alone according to a predetermined threshold probability referred to as a significance level (e.g., p-values, false discovery rate (FDR), q-values, etc. as values less than 0.05, 0.01, 0.001, 0.0001).

As used herein, “percent (%) sequence identity” with respect to a reference polynucleotide or polypeptide sequence is defined as the percentage of nucleic acids or amino acid residues in a candidate sequence that are identical with the nucleic acids or amino acid residues in the reference polynucleotide or polypeptide sequence, after aligning the sequences and introducing gaps, if necessary, to achieve the maximum percent sequence identity, and not considering any conservative substitutions as part of the sequence identity. Alignment for purposes of determining percent nucleic acid or amino acid sequence identity can be achieved in various ways that are within the skill in the art, for instance, using publicly available computer software such as BLAST, BLAST-2, ALIGN, or Megalign (DNASTAR) software. Those skilled in the art can determine appropriate parameters for aligning sequences, including any algorithms needed to achieve maximal alignment over the full length of the sequences being compared.

As used herein, “nucleic acid” or “nucleotide” refers to deoxyribonucleotides or ribonucleotides and polymers thereof in either single-or double-stranded form and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acid as used herein also refers to nucleic acids that have the same basic chemical structure as a naturally occurring nucleic acid. Such analogues have modified sugars and/or modified ring substituents but retain the same basic chemical structure as the naturally occurring nucleic acid. A nucleic acid mimetic refers to chemical compounds that have a structure that is different the general chemical structure of a nucleic acid, but that functions in a manner similar to a naturally occurring nucleic acid. Examples of such analogues include, without limitation, phosphorothioates, phosphoramidates, methyl phosphonates, chiral-methyl phosphonates, 2-O-methyl ribonucleotides, and peptide-nucleic acids (PNAs). The term “oligonucleotide” refers to a relatively short polynucleotide (e.g., less than about 250 nucleotides in length), including, without limitation, single-stranded deoxyribonucleotides, single- or double-stranded ribonucleotides, RNA: DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

As used herein, “optional” or “optionally” means that the subsequently described event or circumstance can or cannot occur, and that the description includes instances where the event or circumstance occurs and instances where it does not. For example, the phrase optionally the composition can comprise a combination means that the composition may comprise a combination of different molecules or may not include a combination such that the description includes both the combination and the absence of the combination (i.e., individual members of the combination).

As used herein, “treatment” (and grammatical variations thereof, such as “treat” or “treating”) refers to clinical intervention in an attempt to alter the natural course of the subject being treated and can be performed either for prophylaxis or during the course of clinical pathology. Desirable effects of treatment include, but are not limited to, preventing occurrence or recurrence of a disease (e.g., lung cancer), alleviation of symptoms, diminishment of any direct or indirect pathological consequences of the disease, preventing metastasis, decreasing the rate of disease progression, amelioration or palliation of the disease state, and remission or improved prognosis. In some embodiments, the treatments described herein are used to delay development of a disease or to slow the progression of a disease. In some instances, the treatment may increase overall survival (OS) (e.g., by about 20% or greater). In some instances, the treatment may increase the progression-free survival (PFS) (e.g., by about 20% or greater). In some instances, the treatment may increase PFS, e.g., by about 5% to about 500%).

As used herein, “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

### DETAILED DESCRIPTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail to avoid obscuring the embodiments.

Also, it is noted that individual embodiments may be described as a process which is depicted as a flowchart, a flow diagram, a data flow diagram, a structure diagram, or a block diagram. Although a flowchart or diagram may describe the operations as a sequential process, many of the operations may be performed in parallel or concurrently. In addition, the order of the operations may be re-arranged. A process is terminated when its operations are completed but could have additional steps not included in a figure. A process may correspond to a method, a function, a procedure, a subroutine, a subprogram, etc. When a process corresponds to a function, its termination may correspond to a return of the function to the calling function or the main function.

### I. INTRODUCTION

CTAs are a category of tumor antigens that are normally expressed in the testes (specifically the male germ cells) and placenta but can also be aberrantly expressed in tumors of different histological origin. Due to their restricted expression patterns, CTAs are considered prognostic biomarkers and targets for immunotherapeutic interventions. In addition, CTA expression is a valuable tool for the selection of subjects likely to respond to treatments directly or indirectly targeting CTA, particularly those leveraging the immunogenic nature of these proteins. More specifically, CTA overexpression drives the initiation, metastasis, and progression of lung cancer, and overall is associated with poor prognosis in lung cancer patients. CTA-based biomarkers can be applied in the diagnosis, monitoring, and treatment of various cancers including lung cancer due to their natural restricted expression patterns and can therefore serve as an additional metric for diagnosing and monitoring cancer. In addition, CTA expression can be a valuable tool for the selection of subjects likely to respond to treatments directly or indirectly targeting CTA, particularly those leveraging the immunogenic nature of these proteins. To increase the efficacy of cancer diagnosis, prognostic prediction, and drug discovery, robust CTA identification and quantitation is needed.

To address these challenges and others, provided herein are diagnostic and therapeutic techniques for the treatment of cancer (e.g., lung cancer) based on the quantification of the summed expression and co-expression of CTAs in cancer to determine cancer testis antigen burden (CTAB), a novel biomarker or biomarker signature of immunotherapy response. For example, provided herein are diagnostic and therapeutic techniques of predicting therapeutic responsiveness, methods of monitoring responsiveness to treatment, methods of selecting a treatment, methods of treatment, and diagnostic kits developed based on CTAB.

### II. DIAGNOSTIC METHODS

Disclosed herein are methods for (i) treating a subject having cancer, (ii) identifying a response to a treatment in a subject having cancer, (iii) identifying a subject having cancer who will benefit from a treatment such as an immunotherapy (e.g., immune checkpoint inhibitors, cancer vaccines, cellular and antibody-based therapies, and the like) alone or in combination with another anti-cancer therapy targeting one or more CTAs, and (iv) selecting a treatment for a subject having cancer based on a CTAB score. The methods described herein are based on the finding that a CTAB score determined from a tumor sample from the subject having cancer may be used to predict the therapeutic efficacy of a treatment such as immunotherapy alone or in combination with another anti-cancer therapy. Moreover, the findings described herein indicate that the CTAB score represents aspects of the tumor microenvironment in the biological sample that are not measured by current standard of care testing. Examples of the current standard of care testing comprises PD-L1 protein expression, PD-L1 gene expression, tumor mutational burden, tumor immunogenic signature, cell proliferation, or any combination thereof.

### A. General Diagnostics

In some instances, the methods described herein are used to identify a response to a treatment in a subject having cancer. The method includes (a) obtaining slides of a biological sample from the subject, where at least one slide is stained, and the remaining slides are unstained. (b) The stained slide(s) are examined for for pathological features associated with cancer to determine whether the subject is cancer positive, indeterminant, or cancer negative. (c) In response to the subject being cancer positive or indeterminant: (i) sequencing reads from the remaining unstained slides are generated from a sequencing assay, (ii) using the sequencing reads, the expression of one or more cancer testis antigen (CTA) genes are measured, (iii) determining a cancer testis antigen burden (CTAB) score based on the measured expression of the one or more genes, (iv) classifying the CTAB score as a high-CTAB score or a low-CTAB score compared to a reference CTAB score, and (v) identifying the subject as either responsive or non-responsive to the treatment. In various embodiments, the high-CTAB score is indicative of responsiveness to the treatment, while the low-CTAB score is not indicative of responsiveness to the treatment. (d) In response to the subject being cancer negative, steps (i)-(v) are not performed.

As used herein, the terms “patient,” and “subject” are used interchangeably and refer to any single animal, more preferably a mammal (including humans and non-human animals such as dogs, cats, horses, rabbits, rats, cows, pigs, sheep, and non-human primates). Thus, the methods described herein are applicable to both human and veterinary disease. In certain embodiments, subjects are “patients,” i.e., living humans that are receiving medical care for a disease or condition. This includes persons with no defined illness who are being investigated for signs of cancer pathology. Patients may have an existing tumor or have been diagnosed with a particular cancer. In some cases, a patient may suffer from one or more types of tumors/cancers simultaneously. For example, the subject can have pancreatic, kidney, renal, pelvic, colorectal, stomach, thymic, head and neck, mesothelial, prostate, cervical, thyroid, adrenal, testicular, breast, uterine, bone, esophageal, lung, liver, bile duct, ovarian, bladder, nervous system, and/or skin cancer. In various embodiments, the cancer is lung cancer, more specifically non-small cell lung cancer (NSCLC).

To determine whether the subject is cancer positive, indeterminant, or cancer negative, a biological sample is obtained from the subject. The biological sample derived from the subject includes, but is not limited to, any cell, tissue, or biological fluid comprising nucleic acids (e.g., DNA and/or RNA). In various embodiments, the biological sample is a tissue sample suspected of being cancerous (e.g., a formalin-fixed and paraffin-embedded (FFPE), a fresh frozen (FF), an archival, a fresh, or a frozen tumor tissue sample) from the subject. The processes for collecting a tissue sample are well known in the art and are routinely conducted by a physician, surgeon, or other medical professional. For example, a variety of biopsies known to those skilled in the art can be used to collect tissue samples from an area suspected to have cancer include needle biopsies, fine-needle aspiration biopsies, punch biopsies, needle core biopsies, incisional biopsies, and excisional biopsies, without limitation. The tissue sample may be non-cancerous, benign, malignant, or pre-malignant.

After collection, the tissue sample may be frozen, formalin fixed, and paraffin embedded so that tissue sections may be obtained. The tissue sections may be obtained by cryosection, or any other method known in the art for sectioning tissues and mounting them onto slides. In various embodiments at least one of the tissue section slides (also referred to as “slides)) are stained, while the remaining slides are unstained. The at least one stained slide may be stained using histological staining techniques, such as hematoxylin and eosin (H&E) staining.

The stained slide(s) may be examined by a board-certified anatomical pathologist to identify any pathological features that may be present in the tissue section. Non-limiting examples of pathological features include nuclear atypia, mitotic activity, tumor necrosis, different patterns of invasion, tumor stroma, non-invasive urothelial carcinoma, invasive urothelial carcinoma, low-grade tumors, high-grade tumors, squamous cell differentiation, glandular differentiation, or any combination thereof. Additionally, the pathologist may assess the adequacy of tumor representation, presence of necrosis or issues with fixation or handling, and quality of tissue preservation. Tissue sections with less than 5% tumor tissue and/or more than 50% necrosis can be excluded from further analysis.

Subjects who are suspected of having cancer are likely to have not yet received cancer treatment(s). When such a subject is diagnosed with cancer, their CTAB score is determined, using methods described herein, to predict the treatment plan their cancer type is most likely to respond to. In so doing, the subject is more likely to receive the correct and/or most appropriate treatment plan earlier in their diagnosis (e.g., more effective intervention), improving their overall survival outcomes and reducing their risk of developing preventable side effects due to incorrect or less effective treatment options. In other instances, the subjects have been diagnosed with cancer and are already receiving cancer treatment(s). Accordingly, the treated subject has their CTAB score determined, using methods described herein, to determine the efficacy of their current treatment plan and whether the treatment plan should remain the same (e.g., subject is responsive to treatment) or if the treatment plan should be changed (e.g., subject is not responsive to treatment). Incorporation of a subject's CTAB score into their treatment plan also serves to prevent disease progression due to the subject receiving less effective treatments.

In response to the subject being cancer positive or indeterminant, a sequencing assay is performed to generate sequencing reads from the remaining unstained slides. The sequencing assay may be DNA-sequencing, RNA-sequencing, or both. DNA and/or RNA may be isolated from the remaining unstained slides and processed for tumor mutational burden (TMB) or gene expression, respectively. TMB (as measured by DNA-sequencing methods known in the art) is a measure that quantifies the total number of genetic mutations (mutations per unit of DNA) within the coding region of a tumor's genome. These mutations can include single nucleotide substitutions, insertions, deletions, or other structural alterations. TMB is often expressed as the number of mutations per mega-base (Mb) of DNA. High TMB has been associated with better responses to immune checkpoint inhibitor therapies. More specifically, tumors with a high mutation burden are more likely to produce neoantigens (newly formed antigens) as a result of the mutations. These neoantigens can trigger an immune response. TMB may be assessed through next-generation sequencing (NGS) technologies, which allow for the comprehensive analysis of the tumor's genomic landscape.

To measure the expression of one or more genes, RNA is isolated from the tissue sections and processed for gene expression. Any suitable method of determining the expression level of a gene (e.g., one or more CTA genes) may be used. For example, the expression of CTAs can be measured by various means known in the art for detecting proteins (e.g., IHC and Western Blot) or nucleic acids such as RNA and DNA (e.g., liquid biopsy, PCR, and sequencing). In the methods described herein, one or more CTAs in unstained tissue sections from a subject being cancer positive or indeterminant can be measured by RNA-sequencing (e.g., using an RNA ACCESS® protocol or TRUSEQ® RIBO-ZERO® protocol (ILLUMINA®), RT-qPCR, qPCR, multiplex qPCR or RT-qPCR, microarray analysis, SAGE, MassARRAY techniques, or any combination thereof.

Various established methods are available for assessing mRNA levels within cells. These include well-known techniques such as RNA sequencing (RNA-seq),, serial analysis of gene expression (SAGE), and diverse nucleic acid amplification assays, exemplified by reverse transcription polymerase chain reaction (RT-PCR, including quantitative RT-PCR or qRT-PCR) utilizing specific complementary primers designed for a predetermined set of genes. Additionally, these methodologies may incorporate steps to quantify the levels of target mRNA in a biological sample. This can involve simultaneous examination of comparative control mRNA sequences, often derived from a “housekeeping” gene like a member of the actin family. Optionally, the sequence of the amplified target cDNA can be determined.

Further, optional, techniques involve protocols for the detection or analysis of mRNAs, including target mRNAs, in tissue or cell samples utilizing microarray technologies. In this approach, test and control mRNA samples from respective tissue samples are reverse transcribed and labeled to generate cDNA probes. These probes are then hybridized to an array of nucleic acids fixed on a solid support. The array is specifically designed so that the sequence and position of each array member are known. For instance, a solid support may be arrayed with genes whose expression correlates with the clinical benefit of treatment, such as immunotherapy combined with a suppressive stromal antagonist. The hybridization of a labeled probe with a particular array member indicates the expression of the corresponding gene in the sample from which the probe originated.

The one or more genes (i.e., the one or more CTA genes) that are measured from the sequencing reads include all or any combination of the genes listed in Tables 1-5.

TABLE 1

Gene
BAGE
CTAG1B (NY-ESO-1)
CTAG2 (LAGE-1A)
GAGE1
GAGE10
GAGE12J
GAGE13
GAGE2
MAGEA1
MAGEA10
MAGEA12
MAGEA3
MAGEA4
MAGEC2
MLANA
SSX2
XAGE1B

TABLE 2

Gene
BAGE
CTAG1B (NY-ESO-1)
CTAG2 (LAGE-1A)
GAGE1
GAGE12J
GAGE13
GAGE2
MAGEA1
MAGEA10
MAGEA12
MAGEA3
MAGEA4
MAGEC2
SSX2

TABLE 3

Gene
MLANA

TABLE 4

Gene
GAGE10

TABLE 5

Gene
XAGE1B

In some instances, the one or more genes is the panel of genes listed in Table 1 (e.g., the 17-gene signature). In some instances, the one or more genes is the panel of genes listed in Table 2 (e.g., the 14-gene signature) and no other genes. In some instances, the one or more genes is the panel of genes listed in Table 3 (e.g., the 1-gene signature) and no other genes. In some instances, the one or more genes is the panel of genes listed in Table 4 (e.g., the 1-gene signature) and no other genes. In some instances, the one or more genes is the panel of genes listed in Table 5 (e.g., the 1-gene signature) and no other genes. One of skill in the art will appreciate that the pattern of expression of a certain subset of the genes in an identified panel correlate more closely with a more robust/predictive CTAB score. Depending on the cancer type, aggression/severity of the cancer, etc., a critical subset of genes can be analyzed. Limiting the analysis to the critical subset of the identified panel may be particularly useful in quick screening assays, whereas the full set or substantially all the genes in the panel would be used for a robust analysis.

Determining the CTAB score is based on the measured expression of the one or more CTA genes (i.e., a panel of CTAs) listed in Tables 1-5. The CTAB score is calculated by measuring the expression of each of the one or more CTAs as a percentile rank (0-100) at the gene level and summing the percentile ranks at the sample level to derive the CTAB score. Measuring expression of each of the one or more CTAs at the gene level involves assessing the amount of mRNA (messenger RNA) produced by a specific gene using one or more of the established methods that are available for assessing mRNA levels within cells from the tissue sections, as described herein. In various embodiments, the sequencing assay used for assessing mRNA levels within the tissue section is RNA-sequencing. Gene expression (e.g., amount/level of mRNA produced be a specific gene) is based on the normalized reads per million (nRMP) or each gene transcript considered in the overall CTAB score. In general, RNA-sequencing procedures generate about 5-40 million reads per sample, where any whole number between 5 million and 40 million is considered. The read count for a gene is determined by summing the reads associated with each feature (e.g., exon) that belong to that gene. In other words, summing the reads that uniquely map to each feature of a gene. Accordingly, the read count is highly dependent on the size (e.g., number of exons), expression level of the gene, read length, and the like. Moreover, the read count may differ among genes with alternative transcripts (e.g., transcripts due to alternative splicing where exons from the same gene are combined into different mRNA transcripts) based on their frequency of occurrence. A read is considered associated with a gene if the read is uniquely mapped to the gene and (i) completely overlaps an exon, (ii) partially overlaps an exon, (iii) maps to a junction between two different exons, or any combination thereof. Summing at the sample level involves summing across the percentile ranks of each CTA detected in the sample.

In some instances, the CTAB score is classified as high or low by comparing it to a reference CTAB score. A reference CTAB score is calculated as a mean or median CTAB score from a reference population having a same tumor or cancer type as the subject. To calculate the reference CTAB score, (i) the CTAB score for all subjects within the reference population is determined, (ii) calculating the mean or median for the CTAB scores determined for all the subjects is determined, and (iii) the mean or median CTAB score is assigned as the reference CTAB score for the given tumor or cancer type. In some instances, the reference CTAB score is determined by summing the gene expression ranks of one or more CTA genes. For example, when 17 genes are used, a reference CTAB score may be between 0-1700 (e.g., 0,1, 1.5, 2, 3, 4, 5, 50, 100, 200, 1000, or 1700). As such, the CTAB score may be any whole integer value or rational number (i.e., decimal) between 0-1700. It would be clear to one of ordinary skill in the art, that the number of genes used to determine a CTAB score may be less than or greater than 17 genes. For example, the number of genes may be one or more, for example, 1, 5, 10, 11, 12, 13, 14, 15, 16, 17, 18, 19, 20, 21, 22, 23, 24, 25, etc. In some instances, the reference population is a population of subjects who have not received a treatment including immunotherapy. In some instances, the reference population is a population of subjects who have not received an immunotherapy prior to collection of the sample to be used for calculating the reference CTAB score. In some instances, the reference population is a population of subjects who are who have not received an anti-cancer treatment. In some instances, the reference population is a population of subjects have not received an anti-cancer treatment prior to collection of the sample to be used for calculating the reference CTAB score. In some instances, the reference population is a population of subjects who are not currently receiving an anti-cancer treatment. In some instances, the reference population is a population of subjects who are currently receiving an anti-cancer treatment. A high CTAB score may be a CTAB score≥a reference CTAB score (e.g., reference mean/median score) and a low-CTAB score is CTAB score<a reference CTAB score (e.g., reference mean/median score).

It should be understood that the relative mean or median value of CTAB scores (i.e., the reference CTAB scores) across different cancer or tumor types can vary considerably, and thus what qualifies as a high/low CTAB score is not uniform across cancer or tumor types. By way of example, colorectal cancer may have a mean CTAB score of 15, whereas NSCLC may have a mean CTAB score of 171. Therefore, the median cutoff for colorectal cancer may be much lower compared to NSCLC. Furthermore, the proportion of subjects with a high/low CTAB score may also vary considerably across different cancer types. For example, 88% of subjects with melanoma may have a high CTAB score, but only 15% of subjects with pancreatic cancer may have a high CTAB score. Furthermore, whether a high/low CTAB score is indicative of responsiveness to a treatment may also be dependent on the cancer type. For example, if the subject has a NSCLC tumor, a high CTAB score is associated with a favorable response to the treatment and a low CTAB score is associated with a less-favorable response to the treatment and a more favorable response to a different treatment.

In various embodiments, the CTAB score for any particular cancer has a weak or nonsignificant correlation with other biomarkers used in standard of care methods. For example, CTAB scores have weak or nonsignificant correlations with PD-L1 protein expression, PD-L1 gene expression, tumor mutational burden, tumor immunogenic signature, cell proliferation, or any combination thereof. In other words, CTAB scores represents aspects of the tumor microenvironment in the tumor sample that are not measured by current standard of care testing. As such, determination and implementation of CTAB scores into cancer diagnostics (i) allows for the identification of subjects that previously would not be considered candidates for immunotherapy and (ii) identifies responders and non-responders to immunotherapy that may otherwise be misclassified based on the traditional treatment selection criteria, namely PD-L1 status.

Based on the determined CTAB score, cancer type, and reference CTAB score, this information is used to identify the subject as responsive to the treatment. In some instances, the high-CTAB score is indicative of responsiveness to the treatment, while the low-CTAB score is not indicative of responsiveness to the treatment. A responder (i.e., responsive) and conversely, non-responder (i.e., not responsive) to treatment or a combination of treatments can be classified in several ways. Moreover, the classification can be dependent on the CTAB score in comparison to the reference CTAB score, the subject's cancer type, the severity of the cancer, whether the subject has previously received treatment for the cancer, or any combination thereof. For example, responders to a treatment or a combination of treatments may be classified based on subjects' whose overall survival (OS) improved by about 20% (e.g., at least about 20%, at least about 25%, at least about 30%, at least about 35%, at least about 40%, at least about 45%, at least about 50%, at least 55%, at least about 60%, at least about 65%, or at least about 70% or more). In another example, responders to treatment or a combination of treatments may be classified based on subjects' whose tumor has shrunk, slowed, or stopped growing. In another example, responders to treatment or a combination of treatments may be classified by a physician or other medical professional using the Response Evaluation Criteria in Solid Tumors (RECIST) v1.1 criteria, which assess tumor burden to determine if a subject is responding to treatment or a combination of treatments. Additional information regarding the RECIST criteria for tumors and therapeutic responsiveness can be found in Eisenhauer, Elizabeth A., et al. “New response evaluation criteria in solid tumors: revised RECIST guideline (version 1.1).” European journal of cancer 45.2 (2009): 228-247. In another example, responders to treatment or a combination of treatments may be classified based on subjects that have a complete response (CR) or partial response (PR). In another example, non-responders to treatment or a combination of treatments may be classified based on subjects that have stable disease (SD), or progressive disease (PD). In any of the aforementioned examples, classification of responders and non-responders to treatment or a combination of treatments may be additionally based on the CTAB score in comparison to the reference CTAB score.

In various embodiments, examination of the stained slide(s) for pathological features associated with cancer reveal that the subject is cancer negative. Accordingly, the methods for obtaining sequencing reads to determine a CTAB score for the subject would not be performed. Moreover, a subject found to be cancer negative would not receive cancer related treatments.

### B. Treatment

In any of the methods described herein, the methods further include administering to the subject an effective amount of a treatment when the CTAB score is indicative of responsiveness. Provided herein is a method of treating a subject having cancer. The method of treating includes initially obtaining sequencing reads from a biological sample from the subject by performing a sequencing assay. As described above, the processes for collecting a biological sample are well known in the art and are routinely conducted by a physician, surgeon, or other medical professional. In various embodiments, the biological sample may be cells, tissue, or biological fluid collected from the subject. More specifically, the biological sample is a tissue/tissue biopsy collected from a cancerous tissue source from the subject. The sequencing reads may be generated by a sequencing assay such as RNA-sequencing for measuring gene expression and/or DNA-sequencing for measuring TMB. The sequencing reads may be used to measure the expression of one or more cancer testis antigen (CTA) genes in the biological sample from the subject having cancer. From the measured expression data, a cancer testis antigen burden (CTAB) score is determined and classified as either a high-CTAB score or a low-CTAB as compared to a reference CTAB score. In response to the CTAB score being a high-CTAB score, the subject is treated with an effective amount of a treatment. In response to the CTAB score being a low-CTAB score, the subject is treated with an effective amount of a different treatment.

In any of the methods described herein, the treatment may be an immune checkpoint inhibitor, or treatment may be a combination of treatments comprising an immune checkpoint inhibitor and additional anti-cancer therapy. In other words, a subject is administered an effective amount of a treatment such as an immunotherapy alone or in combination with any anti-cancer therapy other than the immunotherapy (this includes immunotherapies different from the prior immunotherapy administered in this sentence). In various embodiments, the immune checkpoint inhibitor is, without limitation, pembrolizumab, ipilimumab, nivolumab, atezolizumab, cemiplimab, avelumab, or durvalumab. In various embodiments the immune checkpoint inhibitor is pembrolizumab. In various embodiments, the treatment is pembrolizumab. In various embodiments, the additional anti-cancer therapy comprises surgery, chemotherapy, chemoradiation, anticancer drugs, anti-VEGF therapy, novel cancer treatments, or any combination thereof. In various embodiments, the additional anti-cancer therapy is chemotherapy. In various embodiments, the treatment is pembrolizumab in combination with chemotherapy.

As described herein, “a different treatment” can refer to a different dose/concentration of the same immunotherapy agent or immunotherapy-anticancer therapy combinations given to the subject who is responsive to the treatment. For example, a subject with a low CTAB score being treated with a different treatment may receive a different dose/concentration of an immune checkpoint inhibitor such as pembrolizumab, ipilimumab, nivolumab, atezolizumab, cemiplimab, avelumab, or durvalumab. In other instances, a subject with a low CTAB score being treated with a different treatment may receive a different dose/concentration of a combination of treatments comprising an immune checkpoint inhibitor (e.g., selected from the above examples) and an additional anti-cancer therapy. The additional anti-cancer therapy comprises surgery, chemotherapy, chemoradiation, anticancer drugs, anti-VEGF therapy, novel cancer treatments, or any combination thereof. In various embodiments, the additional anti-cancer therapy is chemotherapy. More specifically, the different treatment given to a subject with a low-CTAB score is a combination of pembrolizumab and chemotherapy.

As used herein, “effective amount” of a compound, for example, an immunotherapy as described herein, or a composition (e.g., pharmaceutical composition) thereof, is at least the minimum amount required to achieve the desired therapeutic or prophylactic result, such as a measurable increase in overall survival (OS) or progression-free survival (PFS) of a particular disease or disorder (e.g., a lung cancer). An effective amount herein may vary according to factors such as the disease state, age, sex, and weight of the individual, and the ability of the antibody to elicit a desired response in the subject. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications, and intermediate pathological phenotypes presenting during development of the disease. An effective amount can be administered in one or more administrations. For purposes provided herein, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more treatments (e.g., therapeutic agents or therapy), and a single treatment may be considered to be given in an effective amount if, in conjunction with one or more other treatments, a desirable result may be or is achieved. For example, an effective amount of an immunotherapy as described herein as a cancer treatment may reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TDP), the response rates (RR), duration of response, and/or quality of life.

The method of treating the subject having cancer may further include generating tissue sections of the tumor tissue sample for examination of pathological features associated with cancer. The tumor tissue sample may be frozen, formalin fixed, and paraffin embedded so that the tissue sections may be obtained. The tissue sections may be obtained by cryosection, or any other method known in the art for sectioning tissues and mounting them onto slides. In various embodiments at least one of the slides are stained, while the remaining slides are unstained. The at least one stained slide may be stained using histological staining techniques, such as hematoxylin and eosin (H&E) staining. The at least one stained slide is examined by a board-certified anatomical pathologist to identify pathological features associated with cancer. Non-limiting examples of pathological features include nuclear atypia, mitotic activity, tumor necrosis, different patterns of invasion, tumor stroma, non-invasive urothelial carcinoma, invasive urothelial carcinoma, low-grade tumors, high-grade tumors, squamous cell differentiation, glandular differentiation, or any combination thereof. Additionally, the pathologist may assess the adequacy of tumor representation, presence of necrosis or issues with fixation or handling, and quality of tissue preservation. Tissue sections with less than 5% tumor tissue and/or more than 50% necrosis can be excluded from further analysis.

The method of treating the subject optionally includes repeating the treatment or the different treatment at later time points to determine the efficacy of the treatment or different treatment. In other embodiments, the method of treating the subject optionally includes repeating the method of identifying a response to a treatment at multiple time points to detect changes in the cancer and/or efficacy of treatment. For example, a CTAB score may be determined using the tissue sample (e.g., tumor sample) from the subject at a first time point. Based on the CTAB score and the cancer type, an effective amount of a treatment is administered to the subject. The treatment may be an immune checkpoint inhibitor (e.g., pembrolizumab, ipilimumab, nivolumab, atezolizumab, cemiplimab, avelumab, or durvalumab) alone or in combination with another anti-cancer therapy (e.g., surgery, chemotherapy, chemoradiation, anticancer drugs, anti-VEGF therapy, as well as other novel treatments). At a second and subsequent time points, another CTAB score may be determined using a different tissue sample (e.g., tumor sample) from the subject. The first and second CTAB scores are compared, and a decrease (e.g., a decrease in the CTAB score of about 0.1, 0.2, 0.3, or greater) in the second CTAB score relative to the first CTAB score may indicate a subject who is responsive to treatment with immunotherapy (e.g., ICIs). In some instances, treating the subject further comprises administering one or more additional doses of the treatment if the subject's second CTAB score is decreased relative to the first CTAB score. Conversely, no change or an increase in CTAB (e.g., an increase in the CTAB score of about 0.1, 0.2, 0.3, or greater) in the second CTAB score relative to the first CTAB score may indicate a subject who is not responsive to treatment with the given immunotherapy. Accordingly, the non-responsive patient has their treatment plan adjusted where they are given an effective amount of a different anti-cancer therapy.

In any of the methods and assays described herein, the first CTAB score is a CTAB score determined from a tumor sample from the subject obtained prior to administration of a first dose of treatment including immunotherapy, as described herein. In other words, the sample may be a baseline sample. In other instances, the first CTAB score is a CTAB score determined from a sample from the subject obtained at a previous time point, wherein the previous time point is following (e.g., minutes, hours, or days following) administration of a first dose of treatment including immunotherapy, as described herein. In other instances, the first CTAB score is a CTAB score determined from a sample from the subject obtained at a previous time point, wherein the previous time point is following administration of a first dose of an anti-cancer therapy for the subject other than immunotherapy (e.g., chemotherapy), as described herein. In other instances, the first CTAB score is a predetermined CTAB score, e.g., a reference CTAB score or a subject specific CTAB score determined previously (e.g., prior to relapse). In some instances, multiple samples are obtained from the same subject at different time points (e.g., prior to and following administration of treatment including immunotherapy).

In any of the methods described herein, treating may further include administering a second treatment to the subject, such as for example, a chemotherapeutic. Additionally, the second treatment may be administered in combination with the immunotherapy (e.g., ICI) therapy to treat the subject with cancer. The second treatment may be administered simultaneously, or sequentially in any order during the entire or portions of the treatment period. Further, the second treatment may be administered within a short time of the immunotherapy (e.g., within about within 0.5, 1, 2, 4, 6, 8, 10, 12, 16, 20, or 24 hours of each other). In some instances, the treatment or combination of treatments may be administered following the same or different dosing regimens. In some cases, one treatment is administered following a scheduled regimen while the other treatment is administered intermittently. In some cases, both treatments are administered intermittently, for example one is administered every day and the other is administered weekly or biweekly.

An immune checkpoint blocker (ICB) or inhibitor (ICI), used synonymously throughout, refers to a molecule that totally or partially reduces, inhibits, interferes with, or otherwise modulates the activity of one or more checkpoint proteins. The immune checkpoint inhibitor interacts with immune checkpoint protein(s) on the surfaces of antigen-presenting cells and T-cells to regulate immune responses. Examples of immune checkpoint proteins that the ICI may interact with include, but are not limited to, A2aR (adenosine A2a receptor); BTLA, B, and T (lymphocyte attenuator); ICOS (inducible T cell co-stimulator); KIR (killer cell immunoglobulinlike receptor); LAG3 (lymphocyte activation gene 3); PD1 (programmed cell death protein 1); CTLA-4 (cytotoxic T-lymphocyte-associated antigen 4); and TIM3 (T cell membrane protein 3). Commonly known checkpoint proteins include CTLA4, PD-1, PD-L1, LAG3, B7-H3, B7-H4, TIM3, CD160, CD244, VISTA, TIGIT, and BTLA. (see, e.g., Pardoll, 2012, Nature Reviews Cancer 12:252-264; Baksh, 2015, Semin Oncol. 2015 June; 42 (3): 363-77).

Immune checkpoint inhibitors can, for example, include antibodies or peptide-like compounds derived from antibodies. Examples of immune checkpoint inhibitors, or immune checkpoint blockade therapeutics, include but are not limited to, an anti-PD-L1 antibody, an antibody against PD-1, an antibody against PD-L2, an antibody against CTLA-4, an antibody against KIR, an antibody against IDO1, an antibody against ID02, an antibody against TIM-3, an antibody against LAG-3, an antibody against OX40R, and an antibody against PS. In some instances, the immune checkpoint inhibitor is humanized monoclonal anti-programmed death ligand 1 (PD-L1) antibody, atezolizumab. In other instances, the immune checkpoint inhibitor is an anti-PD-L1 antibody such as avelumab, durvalumab, KN035, CK-301, AUNP12, CA-170, MPDL3280A (RG7446), MEDI4736 and BMS-936559. In other instances, the immune checkpoint inhibitor is an anti-PD-1 antibody such as pembrolizumab (formerly lambrolizumab or MK-3475), nivolumab (BMS-936558), cemiplimab, spartalizumab, camrelizumab, sintilimab, tislelizumab, toripalimab, Pidilizumab (CT-011), AMP-224, or AMP-514. Further examples of immune checkpoint inhibitor, or immune checkpoint blockade (ICB) therapeutics, include but are not limited to, B7-DC-Fc fusion proteins such as AMP-224, anti-CTLA-4 antibodies such as tremelimumab (CP-675,206) and ipilimumab (MDX-010), antibodies against the B7/CD28 receptor superfamily, anti-Indoleamine (2,3)-dioxygenase (IDO) antibodies, anti-IDO1 antibodies, anti-ID02 antibodies, tryptophan, tryptophan mimetic, 1-methyl tryptophan (1-MT)), Indoximod (D-1-methyl tryptophan (D-1-MT)), L-1-methyl tryptophan (L-1-MT), TX-2274, hydroxyamidine inhibitors such as INCB024360, anti-TIM-3 antibodies, anti-LAG-3 antibodies such as BMS-986016, recombinant soluble LAG-3Ig fusion proteins that agonize MHC class II-driven dendritic cell activation such as IMP321, anti-KIR2DL1/2/3 or anti-KIR) antibodies such lirilumab (IPH2102), urelumab (BMS-663513), anti-phosphatidylserine (anti-PS) antibodies such as Bavituximab, anti-idiotype murine monoclonal antibodies against the human monoclonal antibody for N-glycolil-GM3 ganglioside such as Racotumomab (formerly known as 1E10), anti-OX40R antibodies such as IgG CD134 mAb, anti-B7-H3 antibodies such as MGA271, and small interfering (si) RNA-based cancer vaccines designed to treat cancer by silencing immune checkpoint genes.

In some instances, the treatment is a combination of treatments, such as for example, the combination of any one of the above-mentioned immune checkpoint inhibitors with another anti-cancer therapy. Examples of other anti-cancer therapies include traditional cancer treatments such as surgery, chemotherapy, chemoradiation, anticancer drugs (e.g., cisplatin, carboplatin, oxaliplatin, nedaplatin, triplatin tetranitrate, phenanthriplatin, picoplatin, satraplatin), taxane, anti-VEGF therapy, as well as other novel treatments). These other anti-cancer therapies should be expected to act in an additive or synergistic manner with the immune checkpoint inhibitor therapy. Optionally, the combination of treatments may include two or more of any of the above-mentioned immune checkpoint inhibitors so that multiple checkpoint proteins may be targeted. Optionally, the combination of treatments may include two or more types of immunotherapies, for example, one or more immune checkpoint inhibitors with T-cell adoptive transfer. In some instances, the immunotherapy is immune checkpoint inhibitors targeting one or more CTAs as mono-and combination therapies, as set forth in detail herein. In certain instances, the immune checkpoint therapy is anti-PD-1 immunotherapy (pembrolizumab) alone or in combination with chemotherapy. The immune checkpoint inhibitor or combination of immune checkpoint inhibitors selected as the treatment may be dependent on the type of cancer diagnosed for the subject. For example, a subject diagnosed with non-squamous NSCLC may be given pembrolizumab (e.g., KEYTRUDA®) as a treatment while a subject with melanoma may be given ipilimumab.

Examples of chemotherapy chemoradiation agents (and as applicable non-immune checkpoint therapies) include, but are not limited to, mammalian target of rapamycin (mTOR) inhibitors such as sirolimus (also known as rapamycin), alkylating agents such as thiotepa and cyclosphosphamide, phosphatidylinositol 3-kinase (PI3K) inhibitors such as idelalisib, dual phosphatidylinositol 3-kinase (PI3K)/mTOR inhibitors such as XL765, GDC-0980, BEZ235, latinum analogs such as cisplatin and carboplatin, ribozymes such as a VEGF expression inhibitor, vaccines such as gene therapy vaccines, for example, ALLOVECTIN® vaccine, LEUVECTIN® vaccine, and VAXID® vaccine, alkylating agents such as thiotepa and cyclosphosphamide, alkyl sulfonates such as busulfan, improsulfan and piposulfan, nitrogen mustards such as chlorambucil, chlornaphazine, and chlorophosphamide, nitrosoureas such as carmustine, chlorozotocin, fotemustine, lomustine, nimustine, and ranimnustine, antibiotics such as aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, and doxorubicin, platinum agents such as cisplatin, oxaliplatin, and carboplatin, retinoids such as retinoic acid, serine-threonine kinase inhibitors such as rapamycin, farnesyltransferase inhibitors such as lonafarnib, cytotoxic agents such as radioactive isotopes, growth inhibitory agents, and pharmaceutically acceptable salts, acids or derivatives of any of the above as well as combinations of two or more of the above or the like.

The treatments described herein may be formulated into one or more pharmaceutical compositions for delivery into a subject via any route of administration. The route of administration can be any administration pathway known in the art, including but not limited to parenteral, aerosol, nasal, oral, transmucosal, transdermal, intraparenchymal injection, intravenous, intrathecal, intramuscular, intracisternal, or any combination thereof depending on whether local or systemic treatment is desired. The treatments may be suitably administered to the subject at one time or over a series of treatments. For repeated administrations over several days or longer, depending on the condition, the treatment would generally be sustained until a desired suppression of disease symptoms occurs. An initial higher loading dose, followed by one or more lower doses may be administered. However, other dosage regimens may be useful. Immunotherapies described herein can be administered in cyclic administration routines as understood in the art-for example for 2, 3, 6, 12, 15, 18, 24, 30, 48, 60, or more continual weeks followed by a rest period of 1, 2, 3, 4, 5, 6, 7, or more weeks. In some instances, an immunotherapy as described herein is administer as 200 mg every 3 weeks (Q3W)—after dilution as an intravenous infusion over 30 minutes. In another instance, an immunotherapy as described herein is administer as 400 mg every 6 weeks (Q6W)—after dilution as an intravenous infusion over 30 minutes. The immunotherapy may be administered prior to chemotherapy when given on the same day. The immunotherapy may be administered until disease progression, unacceptable toxicity, or up to 24 months. In other instances, immunotherapy as described herein is administered in accordance with a package insert. In other instances, immunotherapy as described herein and effective doses for any of the administration methods described herein can be extrapolated from dose-response curves derived from in vitro or animal model test systems. The progress of the treatment(s) may be monitored by conventional techniques and assays.

Also provided herein is a method for identifying a subject having cancer who will benefit from a treatment comprising immunotherapy alone or in combination with another anti-cancer therapy targeting one or more CTAs. To identify a subject responsive to the treatment, the methods disclosed for identifying a response to a treatment are used. For example, a tumor sample is obtained from the subject and a CTAB score is determined. The CTAB score is determined by measuring the expression of one or more of the CTA genes in Tables 1-5 as a percentile rank at a gene level and summing the percentile ranks at a sample level to derive the CTAB score. The one or more genes include MAGEA10, MAGEA4, GAGE12J, GAGE2, GAGE1, GAGE13, SSX2, CTAG1B, CTAG2, BAGE, MAGEC2, MAGEA1, MAGEA12, MAGEA3, MLANA, XAGE1B, GAGE10, or any combination thereof. When the CTAB score is indicative of responsiveness, a treatment comprising administering an effective amount of an immunotherapy alone or in combination with another anti-cancer therapy is given. In some instances, the treatment includes immunotherapy (e.g., immune checkpoint inhibitors) alone or in combination with another anti-cancer therapy other than the immunotherapy (e.g., chemotherapy or an immunotherapy different from the prior immunotherapy in this sentence, e.g., immune checkpoint inhibitors). In other instances, the treatment includes anti-cancer therapy other than immunotherapy (e.g., chemotherapy).

In some instances, the identified subject has NSCLC and a high-CTAB score (e.g., greater than or equal to a reference CTAB score for NSCLC) indicates the subject will respond to the treatment (or the combination of treatments) and a low CTAB score is predictive of the subject not being responsive to the treatment (or the combination of treatments). In various embodiments, for example, when the subject has NSCLC, the methods of identifying a response to a treatment indicate that a high CTAB score is predictive of the subject being responsive to pembrolizumab monotherapy and a low CTAB score is predictive of the subject not being responsive to pembrolizumab monotherapy. Instead, the low-CTAB score predicts the subject is more likely to be responsive to a combination of pembrolizumab and chemotherapy.

By way of example, subjects with NSCLC are treated with pembrolizumab monotherapy. Of the subjects with NSCLC receiving pembrolizumab monotherapy, those with a high CTAB score have a higher or significantly higher survival probability compared to those subjects with NSCLC receiving pembrolizumab with a low CTAB score. This indicates that subjects with NSCLC and a high CTAB score are more likely to respond to pembrolizumab monotherapy. In various embodiments, subjects with NSCLC are treated with a combination of pembrolizumab and chemotherapy. Of the subjects with NSCLC receiving pembrolizumab and chemotherapy, those with a high CTAB score have a lower or significantly reduced survival probability compared to those subjects with NSCLC receiving pembrolizumab and chemotherapy with a low CTAB score. This indicates that subjects with NSCLC and a low CTAB score are more likely to respond to a combination therapy comprising pembrolizumab and chemotherapy.

By way of example, in various embodiments, subjects with non-squamous NSCLC are treated with pembrolizumab monotherapy. Of the subjects with non-squamous NSCLC receiving pembrolizumab monotherapy, those with a high CTAB score have a higher or significantly higher survival probability compared to those subjects with non-squamous NSCLC receiving pembrolizumab with a low CTAB score. This indicates that subjects with non-squamous NSCLC and a high CTAB score are more likely to respond to pembrolizumab monotherapy. In various embodiments, subjects with non-squamous NSCLC are treated with a combination of pembrolizumab and chemotherapy. Of the subjects with non-squamous NSCLC receiving pembrolizumab and chemotherapy, those with a high CTAB score have a lower or significant reduced survival probability compared to those subjects with non-squamous NSCLC receiving pembrolizumab and chemotherapy with a low CTAB score. This indicates that subjects with non-squamous NSCLC and a low CTAB score are more likely to respond to a combination therapy comprising pembrolizumab and chemotherapy.

By way of example, in various embodiments, subjects with NSCLC and a high CTAB score receive pembrolizumab monotherapy. These subjects have a higher survival probability compared to subjects with NSCLC and a high CTAB score receiving a combination of pembrolizumab and chemotherapy. Furthermore, of the subjects with NSCLC and high CTAB score, more of these subjects are responsive to pembrolizumab monotherapy compared to subjects with NSCLC and high CTAB score receiving the combination of pembrolizumab and chemotherapy. Moreover, subjects with NSCLC and a low CTAB score receive pembrolizumab monotherapy. These subjects have a lower or significantly reduced survival probability compared to subjects with NSCLC and a low CTAB score receiving a combination of pembrolizumab and chemotherapy.

Also provided herein is a method for selecting a treatment for a subject having cancer, wherein the cancer is NSCLC. The methods disclosed for identifying a response to a treatment are used. A CTAB score from a tumor sample from the subject is determined, the subject is identified as one who will respond to a treatment including immunotherapy (e.g., immune checkpoint inhibitors) based on the CTAB score being at or above a reference CTAB score for NSCLC. Accordingly, either an immunotherapy alone or in combination with another anti-cancer therapy other than immunotherapy (e.g., chemotherapy or an immunotherapy different from the prior immunotherapy in this sentence, e.g., immune checkpoint inhibitors) is selected for the subject based on the CTAB score. Additionally or alternatively, a CTAB score from a tumor sample from the subject is below the reference CTAB score, indicating the subject will not be responsive to the treatment. Accordingly, a different therapy is selected comprising a different immunotherapy alone or in combination with an anti-cancer therapy for the subject other than immunotherapy given to the subject who is responsive.

In any of the methods described herein, the method may further include generating a report, e.g., an electronic, web-based, or paper report, to the subject or to another person or entity, a healthcare provider such as a caregiver, a physician, an oncologist, a hospital, or clinic, a third-party payor, an insurance company, a pharmaceutical or biotechnology company, or a government office. In some instances, the report comprises output from the method which comprises evaluation of the determined CTAB score and/or cancer type. For example, the report may be generated to summarize the key findings (e.g., CTBA score(s), whether the subject may benefit from treatment, selected treatment(s), etc.) of the methods and assays in a structured and clear report format. The report may further include information on the experimental design, methods used, references (e.g., the reference CTAB score used for a particular tumor or cancer type), significant genes or pathways, and any relevant clinical correlations.

### III. ASSAY SYSTEM

Disclosed herein is an assay system comprising components useful for the diagnosis and/or monitoring of subjects. The assay system comprises nucleic acid probes that comprise complementary nucleic acid sequences to at least 5, 10, or 50 nucleic acid sequences of one or more genes selected from Tables 1-5 so as to measure the expression level (e.g., mRNA level) of the one or more genes. The assay system may comprise additionally or alternatively antibodies or antigen-binding fragments of antibodies that target polypeptides encoded by the one or more genes selected from Tables 1-5 so as to measure the expression level (e.g., protein level) of the one or more genes. In some instances, the expression level is the RNA level. In some instances, the expression level is the protein level. In some instances, the expression level includes RNA and protein levels. In some instances, an assay system for predicting patient response or outcome to treatment such as immunotherapy for cancer comprises binding ligands that specifically detect polypeptides encoded by the one or more genes selected from Tables 1-5. Exemplary reagents or molecules which specifically bind the one or more genes (gene or polypeptide encoded by the gene), e.g., binding ligand, include but are not limited to antibodies, aptamers and antibody derivatives or fragments. In some instances, the assay system may further comprise an assay surface comprising a chip, array, fluidity card, micro-well plate, or a combination thereof.

### IV. KITS

Disclosed herein is a kit comprising materials useful for the treatment, diagnosis, and/or monitoring of subjects. In some instances, the kit includes one or more reagents for identifying a subject having cancer (e.g., lung cancer) who may benefit from a treatment including immunotherapy as described herein, by determining a CTAB score as described herein, from a sample (e.g., a tissue sample) from the subject. In some instances, the kit further includes one or more reagents for determining the CTAB score from a sample. Optionally, the kit may further include instructions to use the kit to select an anti-cancer treatment such as immunotherapy provided herein for treating a cancer if the CTAB score determined from the sample from the subject is at or above a reference CTAB score. In another instance, the instructions are to use the kit to select an anti-cancer treatment other than an immunotherapy, if the CTAB score determined from the sample from the subject is below a reference CTAB score. In other instances, the kits may include instructions to use the kit to monitor and/or assess the response of a subject having a cancer to treatment such as immunotherapy as described herein.

In some instances, the kit includes one or more reagents comprising one or more nucleic acids. The one or more nucleic acids may be attached to a solid support. In some instances, the one or more nucleic acids comprise a detectable label. In some instances, the one or more nucleic acids are at least 90, 95, 98, 99, or 100% identical to a continuous nucleotide sequence comprising at least 10, 20, 25, 50, 75, 100, 150, or 200 nucleotides (or sequence complementary to the continuous nucleotide sequence comprising at least 10, 20, 25, 50, 75, 100, 150, or 200 nucleotides) within at least one or more genes set forth in Tables 1-4. In some instances, the one or more nucleic acids are at least 5 nucleotides in length and are at least 90, 95, 98, 99, or 100% identical to a continuous 5 nucleotide sequence (or sequence complementary to the continuous 5 nucleotide sequence) within at least one or more genes set forth in Tables 1-4.

In some instances, the kit further includes packaged together a treatment such as immunotherapy as described herein in a pharmaceutically acceptable carrier and a package insert indicating that the treatment is for treating a subject with a cancer as described herein based on a CTAB score determined from a sample from the subject. The kit may include, for example, a container and a label or package insert on or associated with the container. Suitable containers include, for example, bottles, vials, syringes, and the like. The container may be formed from a variety of materials such as glass or plastic. The container holds or contains a composition comprising the treatment, e.g., cancer medicament as the active agent and may have a sterile access port (e.g., the container may be an intravenous solution bag or a vial having a stopper pierceable by a hypodermic injection needle). In some instances, the kit may further include a second container comprising a pharmaceutically acceptable diluent buffer, such as bacteriostatic water for injection, phosphate-buffered saline, Ringer's solution, and/or dextrose solution. The kit may further include other materials desirable from a commercial and user standpoint, including other buffers, diluents, filters, needles, and syringes.

The kit described herein may have a number of instances. In one instance, the kit includes a container, a label on the container, and a composition contained within the container, where the composition includes one or more polynucleotides that hybridize to a complement of a gene listed herein (e.g., a set of genes set forth in any one of Tables 1-4) under stringent conditions, and the label on said container indicates that the composition can be used to evaluate the presence a set of genes listed herein (e.g., a set of genes set forth in any one of Tables 1-4) in a sample, and where the kit includes instructions for using the polynucleotide(s) for evaluating the presence of the genes' RNA or DNA in a particular sample type.

For oligonucleotide-based kits, the kit can include, for example: (1) an oligonucleotide, e.g., a detectably labeled oligonucleotide, which hybridizes to a nucleic acid sequence encoding a protein or (2) a pair of primers useful for amplifying a nucleic acid molecule. The kit can also include, e.g., a buffering agent, a preservative, or a protein stabilizing agent. The kit can further include components necessary for detecting the detectable label (e.g., an enzyme or a substrate). The kit can also contain a control sample or a series of control samples that can be assayed and compared to the test sample. Each component of the kit can be enclosed within an individual container and all of the various containers can be within a single package, along with instructions for interpreting the results of the assays performed using the kit.

The kit provided herein may also include information, for example in the form of a package insert, indicating that the treatment is used for treating a cancer (e.g., lung cancer) based on a CTAB score determined from a sample from a subject as described herein. The insert or label may take any form, such as paper or on electronic media such as a magnetically recorded medium (e.g., floppy disk), a CD-ROM, a Universal Serial Bus (USB) flash drive, Uniform Resource Locator (URL), QR-Code, and the like. The label or insert may also include other information concerning treatments and dosage forms in the kit.

### V. EXAMPLES

The examples below are intended to further illustrate certain aspects of the methods and compositions described herein and are not intended to limit the scope of the claims.

Methods and assays are described herein for predicting ICI response and survival in subjects with tumors across multiple histologies, and particularly in NSCLC. In addition, the following examples revealed that CTA expression is reliably measured using comprehensive genomic and immune profiling (CGIP) in solid tumors. CTAs have a diverse range of expression across tumors and are co-expressed within tumors. CTAB is a novel biomarker of CTA expression and co-expression that quantifies the degree to which these restrictively expressed, immunogenic antigens are present in the tumor microenvironment. Importantly, CTAB is independent from PD-L1 expression and TMB, indicating that CTAB reveals aspects of tumor immunogenicity not measured by current standard of care testing and allows for the identification of subjects not considered candidates for immunotherapy. Improved overall survival for high CTAB NSCLC subjects treated with pembrolizumab monotherapy provide a unique underlying aspect of lung cancer biology that portends a CTA-driven immunogenic environment. Furthermore, the findings show that CTAB identifies responders and non-responders to immunotherapy that may otherwise be misclassified based on the traditional treatment selection criteria, namely PD-L1 status. Finally, the higher rate of stable disease but a non-significant impact on overall survival observed in the CTAB-high subjects treated with combination pembrolizumab and chemotherapy, demonstrate that chemotherapy or some common characteristic of the combination therapy-treated cohort, influences the immunogenic effects of CTA expression resulting in a smaller proportion of partial and complete response and a larger proportion of stable disease.

### A. Techniques for Characterizing CTAB

### 1. Subjects and Clinical Data

Two separate cohorts were assembled from real-world subject samples upon which comprehensive genomic and immune profiling (CGIP) was performed during routine clinical care using the OmniSeq Insight assay. The first cohort was a discovery cohort of clinically tested solid tumors used for development of the immunogenic signature and to establish a low-and high-CTAB cutoff (Table 6). A total of 5,250 subjects were included based on the following criteria: (1) availability of high-quality gene expression data from samples clinically tested by a CLIA (Clinical Laboratory Improvement Amendments) approved targeted RNA-seq assay; (2) samples that pass clinically approved tissue, nucleic acid and sequencing quality control metrics; (3) samples that have less than 50% necrosis and at least 5% tumor purity; and (4) availability of PD-L1 immuno-histochemistry (IHC) and tumor mutational burden (TMB).

TABLE 6
Discovery cohort description

| Variable | Group | N | % of Cohort |
|---|---|---|---|
| Tumor Type | Adrenal Gland Cancer | 4 | 0.1% |
| | Bladder Cancer | 80 | 1.5% |
| | Brain and Nervous System Cancer | 55 | 1.1% |
| | Breast Cancer | 487 | 9.3% |
| | Cervical Cancer | 49 | 0.9% |
| | Colorectal Cancer | 603 | 11.5% |
| | Esophageal Cancer | 159 | 3.0% |
| | Head and Neck Cancer | 144 | 2.7% |
| | Kidney and Renal Pelvis Cancer | 42 | 0.8% |
| | Liver and Bile Duct Cancer | 81 | 1.5% |
| | Lung Cancer | 2264 | 43.1% |
| | Melanoma | 129 | 2.5% |
| | Mesothelioma | 19 | 0.4% |
| | Ovarian Cancer | 270 | 5.1% |
| | Pancreatic Cancer | 216 | 4.1% |
| | Prostate Cancer | 153 | 2.9% |
| | Sarcoma | 192 | 3.7% |
| | Stomach Cancer | 101 | 1.9% |
| | Testicular Cancer | 4 | 0.1% |
| | Thymic Cancer | 11 | 0.2% |
| | Thyroid Cancer | 32 | 0.6% |
| | Uterine Cancer | 155 | 3.0% |
| Cancer Type | Metastatic | 2381 | 45.4% |
| | Primary | 2819 | 53.7% |
| | Recurrent | 45 | 0.9% |
| | No Data | 5 | 0.1% |

| | | |
|---|---|---|
| All Samples | 5250 | 100.0% |

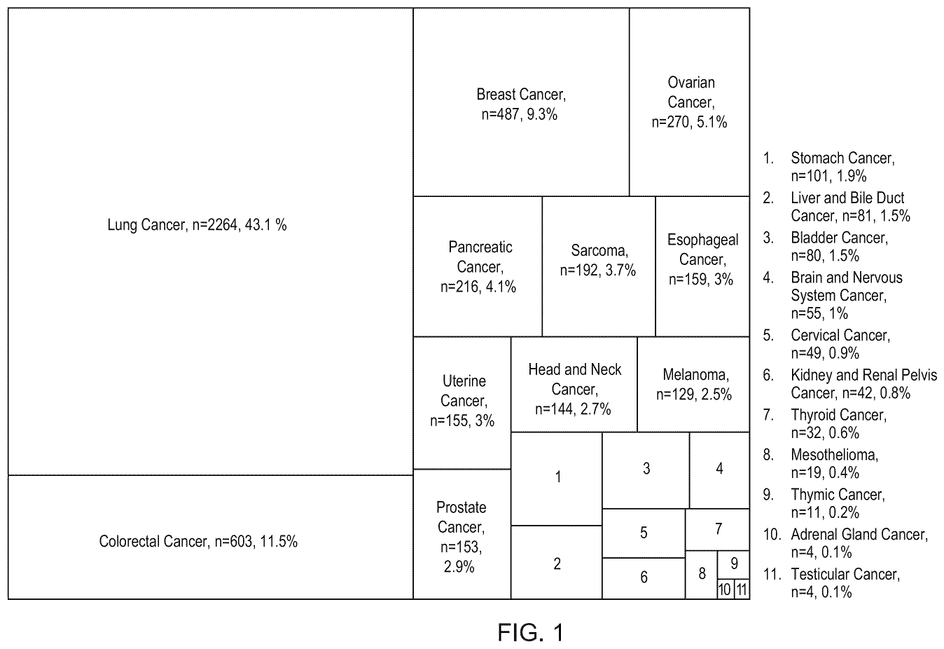

CPIG was performed on 5250 FFPE tumors representing 22 histologic types, assessing expression levels of 395 immune genes and >500 tumor-associated genes. As shown in FIG. 1, the DC primarily consists of lung cancer (43.1%) followed by colorectal (11.5%), breast (9.3%), and ovarian cancer (5.1%), with other cancer types in lesser percentages. Targeted RNA-seq was performed on the 5250 FFPE tumors. From the gene expression data of the DC, three gene expression signatures were calculated: cell proliferation (CP), tumor immunogenic signature (TIGS), and cancer testis antigen burden (CTAB). PD-L1 status of each tumor was assessed by IHC, and tumor mutational burden (TMB) was calculated.

Supporting whole transcriptome data used to duplicate the derivation of CTAB biomarker in the discovery cohort was obtained from The Cancer Genome Atlas (TCGA). The TCGA included 21,975 tumors across 22 tumor types (Table 7).

TABLE 7
The Cancer Genome Atlas (TCGA) cohort description.

| Variable | Group | N | % of Cohort |
|---|---|---|---|
| Age | [10,20) | 95 | 0.4% |
| | [20,30) | 743 | 3.4% |
| | [30,40) | 1573 | 7.2% |
| | [40,50) | 2942 | 13.4% |
| | [50,60) | 4758 | 21.7% |
| | [60,70) | 6066 | 27.6% |
| | [70,80) | 4231 | 19.3% |
| | [80,90) | 1352 | 6.2% |
| | [90,100) | 164 | 0.8% |
| | No Data | 51 | 0.2% |
| Sex | Female | 11333 | 51.6% |
| | Male | 10642 | 48.4% |
| Cancer Type | Adrenal Gland Cancer | 79 | 0.4% |
| | Bladder Cancer | 693 | 3.2% |
| | Brain and Nervous System Cancer | 1315 | 6.0% |
| | Breast Cancer | 3045 | 13.9% |
| | Cervical Cancer | 379 | 1.7% |
| | Colorectal Cancer | 1390 | 6.3% |
| | Esophageal Cancer | 364 | 1.7% |
| | Head and Neck Cancer | 1196 | 5.4% |
| | Kidney and Renal Pelvis Cancer | 2854 | 13.0% |
| | Liver and Bile Duct Cancer | 1387 | 6.3% |
| | Lung Cancer | 2697 | 12.3% |
| | Melanoma | 529 | 2.4% |
| | Mesothelioma | 87 | 0.4% |
| | Ovarian Cancer | 365 | 1.7% |
| | Pancreatic Cancer | 253 | 1.2% |
| | Prostate Cancer | 1292 | 5.9% |
| | Sarcoma | 349 | 1.6% |
| | Stomach Cancer | 898 | 4.1% |
| | Testicular Cancer | 209 | 1.0% |
| | Thymic Cancer | 150 | 0.7% |
| | Thyroid Cancer | 1510 | 6.9% |
| | Uterine Cancer | 934 | 4.3% |

| | | |
|---|---|---|
| All Samples | 21975 | 100.0% |

The second cohort was a retrospective NSCLC cohort for which response to ICI therapy and overall survival was available. A total of 250 tumors were used from subjects with NSCLC (213 non-squamous tumors and 37 squamous tumors) treated with ICIs (Table 8).

TABLE 8
Retrospective cohort description

| Variable | Group | N | % of Cohort |
|---|---|---|---|
| Age | [30,40) | 1 | 0.4% |
| | [40,50) | 5 | 2.0% |
| | [50,60) | 40 | 16.0% |
| | [60,70) | 105 | 42.0% |
| | [70,80) | 75 | 30.0% |
| | [80,90) | 23 | 9.2% |
| | [90,100) | 1 | 0.4% |
| Sex | Female | 136 | 54.4% |
| | Male | 114 | 45.6% |
| Tumor Type | NSCLC | 250 | 100.0% |
| Histology Type | Non-Squamous | 213 | 85.2% |
| | Squamous | 37 | 14.8% |
| Treatment Group | pembro. + chemo. | 148 | 59.2% |
| | pembrolizumab | 102 | 40.8% |

| | | |
|---|---|---|
| All Samples | 250 | 100.0% |

The inclusion criteria for this retrospective cohort study required subjects to have received treatment with an FDA-approved ICI as of November 2017, and to have follow-up and survival data from the first ICI dose. In addition, subjects had to have an evaluable response based on Response Evaluation Criteria in Solid Tumors (RECIST) v1.1 criteria. Subjects who had a complete response (CR) or partial response (PR) based on RECIST v1.1 criteria were classified as responders, while those who had stable disease (SD), or progressive disease (PD) were classified as non-responders. However, the duration of response was not available for all subjects and was not included in the final analysis.

### 2. Restricted Expression of CTAs in Testis Tissue

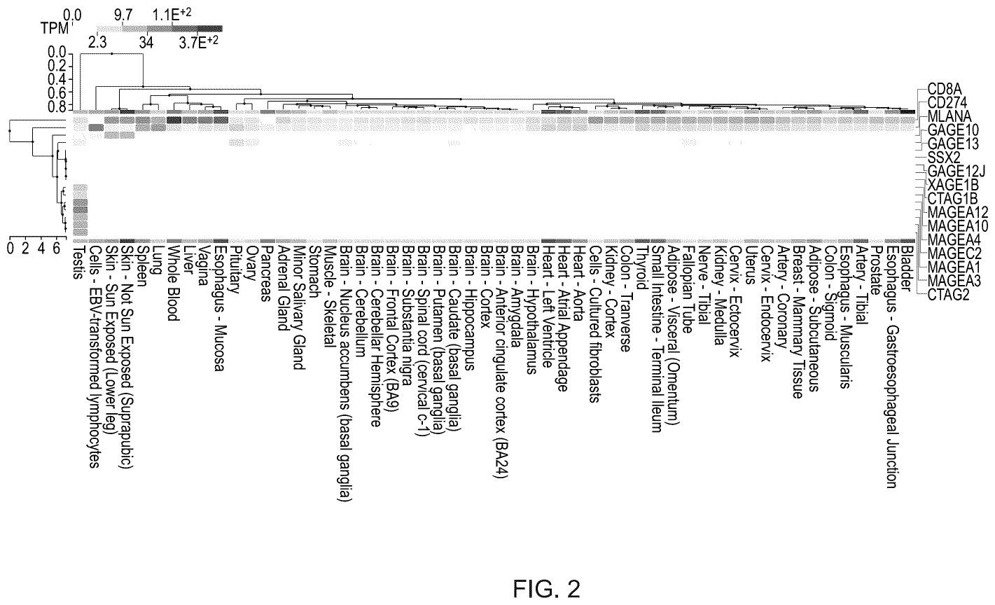

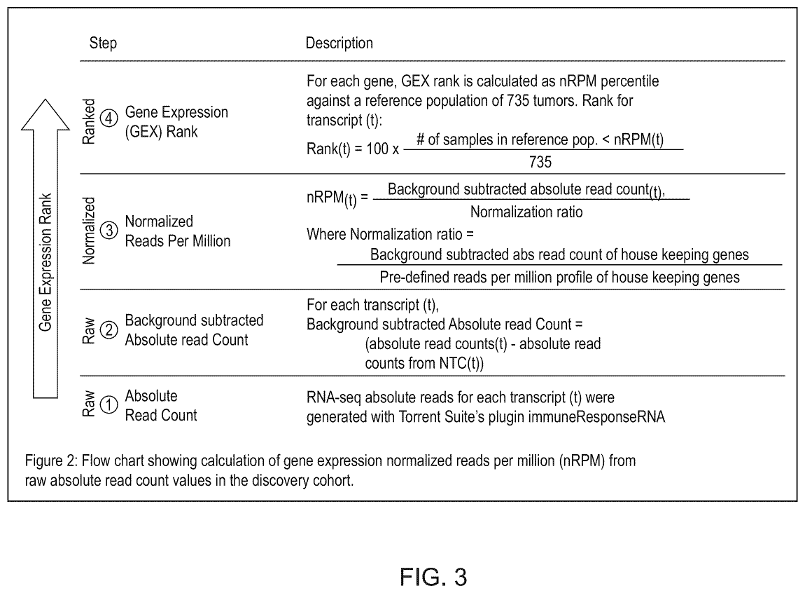

Whole-transcriptome tissue type-specific CTA expression data for normal tissue was obtained from GTEx (Genotype-Tissue Expression). Data from the tissue gene expression database GTEx was profiled in order to evaluate the expression patterns of CTAs across diverse tissue types. Among the 54 tissue types profiled, robust CTA expression was observed only in testis tissue (FIG. 2). Additionally, immune-associated genes such as CD8A and PD-L1 (CD274) were expressed in most of the tissue types profiled, with the notable exception of the testes, ovaries, and CNS, in line with the immuno-privileged nature of those tissue types (FIG. 2).

### 3. CTA Co-Expression

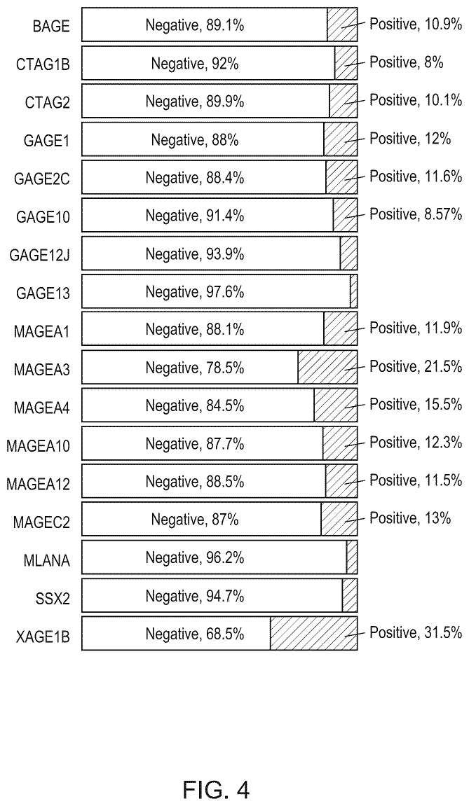

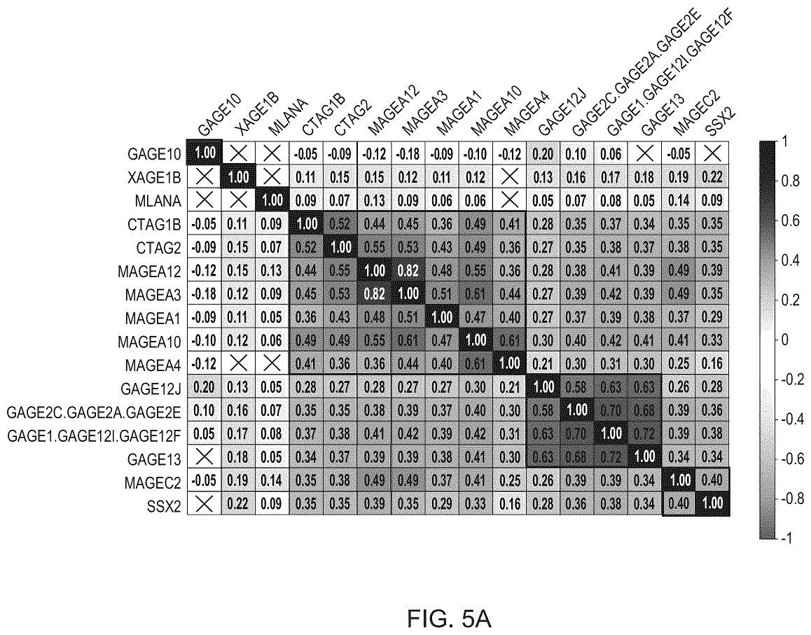

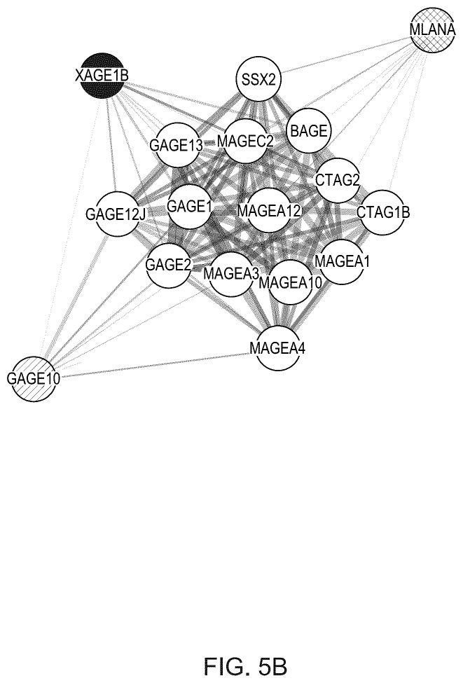

RNA-seq of the discovery cohort was used to determine the expression of 17 CTA genes in 5,250 real-world solid tumor tissues. The method for calculating gene expression normalized reads from raw absolute read count and calculating the Gene Expression (GEX) rank is shown in FIG. 3. As shown in FIG. 4, the CTAs measured were BAGE, CTAG1B, CTAG2, GAGE1, GAGE2, GAGE10, GAGE12J, GAGE13, MAGEA1, MAGEA3, MAGEA4, MAGEA10, MAGEA12, MAGEC2, MLANA, SSX2, and XAGE1B. Positive CTA expression prevalence ranged from 3% (GAGE13) to 31.5% (XAGE1B) across the 5250 tumors studied. The Co-expression pattern of the 17 CTAs across the entire 5250 tumors in the discovery cohort is shown in FIG. 5. Co-expression of CTAs was observed across several histologies, with the highest levels of co-expression observed within the MAGEA and GAGE families.

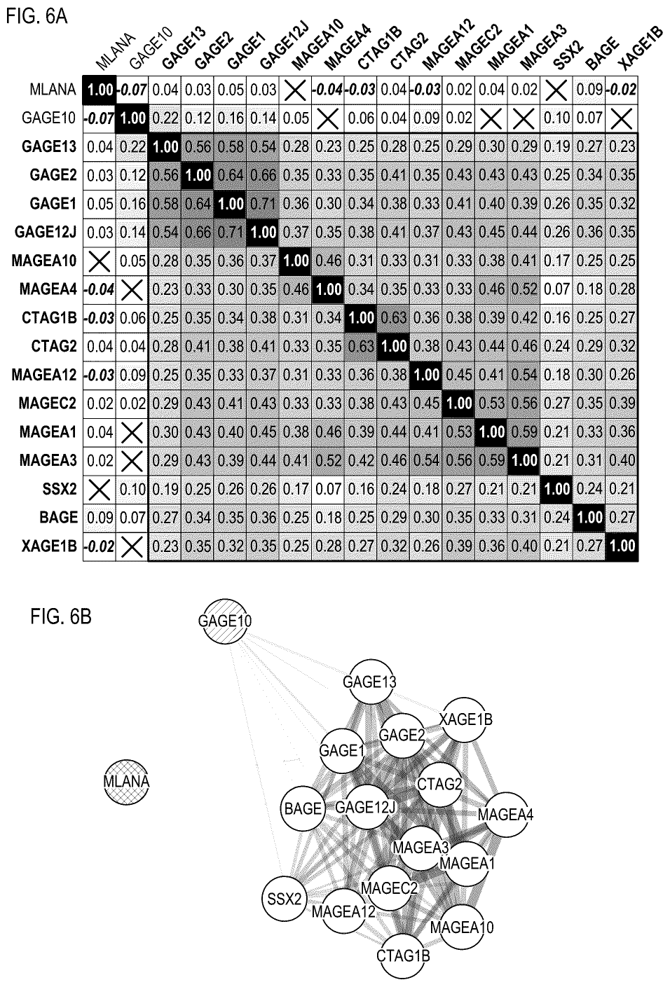

High levels of CTA co-expression were observed in multiple tumor types, quantified by significant positive Spearman correlation (ρ), between the 16 CTA (FIGS. 5A and 5B). In the discovery cohort, 13 of the 16 profiled CTA were significantly co-expressed with one another, and the remaining three were expressed largely independently of the others, resulting in four CTA expression groups: 1) MAGEA10, MAGEA4, GAGE12J, GAGE2, GAGE1, GAGE13, SSX2,CTAG1B, CTAG2, MAGEC2, MAGEA1, MAGEA12, and MAGEA3 [mean ρ_{group}=0.37, mean ρₐₗₗ=0.28, max ρ_{any}=0.72]; 2) MLANA [mean ρₐₗₗ=0.08, max ρₐₗₗ=0.10]; 3) XAGE1B [mean ρₐₗₗ=0.14, max ρ_{any}=0.20]; and 4) GAGE10 [mean ρₐₗₗ=0.02, max ρ_{any}=0.27] (FIGS. 5A and 5B). Similar co-expression was observed in the TCGA cohort (FIGS. 6A and 6B), except only three CTA expression groups were observed: 1) GAGE13, GAGE2, GAGE1, GAGE12J, MAGEA10, MAGEA4, CTAG1B, CTAG2, MAGEA12, MAGEC2, MAGEA1, MAGEA3 SSX2, BAGE, and XAGE1B; 2) MLANA; and 3) GAGE10.

### 4. Cance Testis Antigen Burden in Cancer

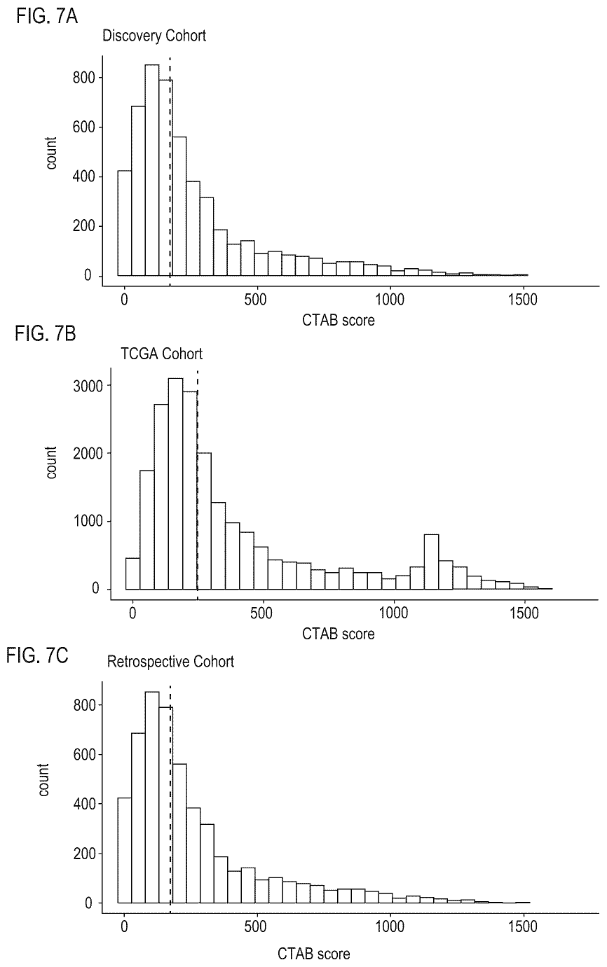

Based on the significant co-expression of CTAs in tumors; to capture the combined CTA expression, the novel biomarker know as cancer testis antigen burden (CTAB) was developed. CTAB was calculated, for each sample, cohort and tumor type, by summing the gene expression ranks of all 17 CTAs, giving an integer value between 0 and 1700. The results of each cohort CTAB composition are shown below in Table 9. As shown in FIG. 7, the three cohorts each demonstrated overlapping single-peak, left-skewed CTAB distribution curves centered at CTAB values of 171 for the discovery cohort (FIG. 7A), 247 for the TCGA cohort (FIG. 7B), and 193 for the retrospective cohort (FIG. 7C). The discovery cohort median CTAB of 171 was used to classify all three cohorts into high-and low-CTAB groups in downstream analysis.

TABLE 9
Cohort CTAB composition

| | | Median | N Positive | N Negative |
|---|---|---|---|---|
| Cohort | N | CTAB | (CTAB ≥ 171) | (CTAB < 171) |
| Discovery | 5250 | 171 | 2628 | 2622 |
| Cohort | | | | |
| TCGA | 21975 | 247 | 14748 | 7227 |
| Cohort | | | | |
| Retrospective | 250 | 193 | 148 | 102 |
| Cohort | | | | |

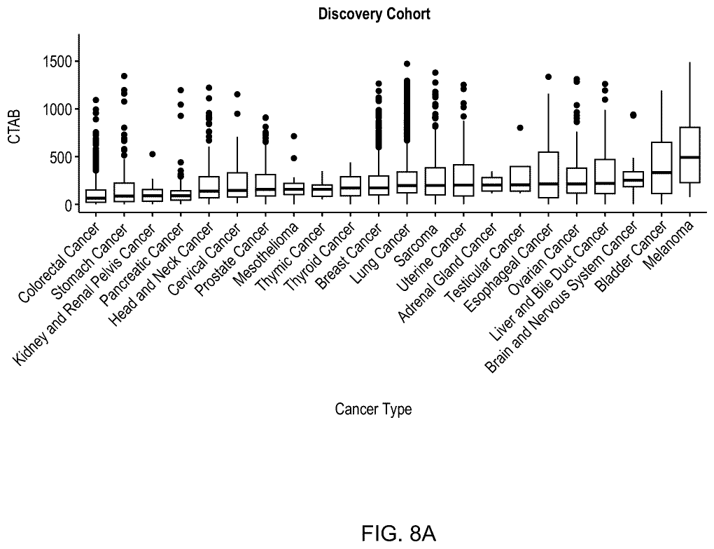

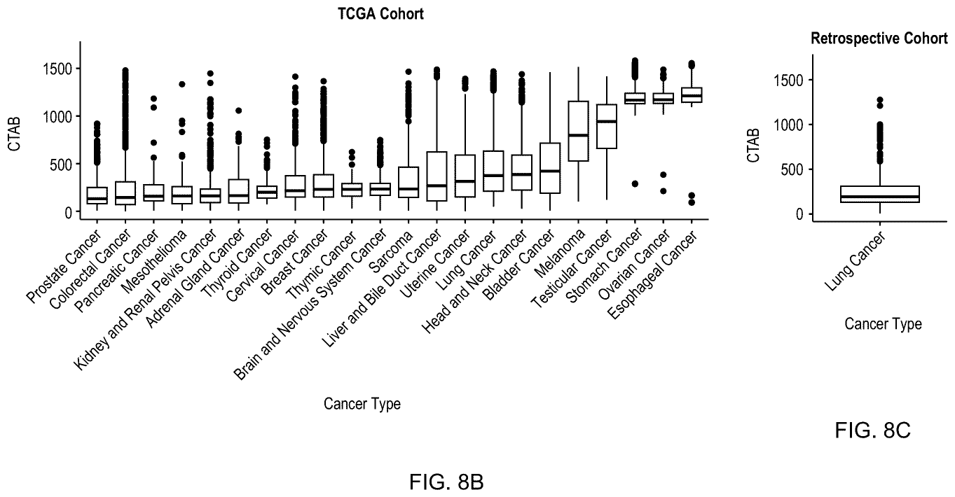

As shown in FIGS. 8A-8C, when grouped by tumor types and ordered by median CTAB, the CTAB distributions for the 22 tumor types within the three cohorts (discovery cohort (FIG. 8A); TCGA cohort (FIG. 8B); and Retrospective cohort (FIG. 8C)) were comparable.

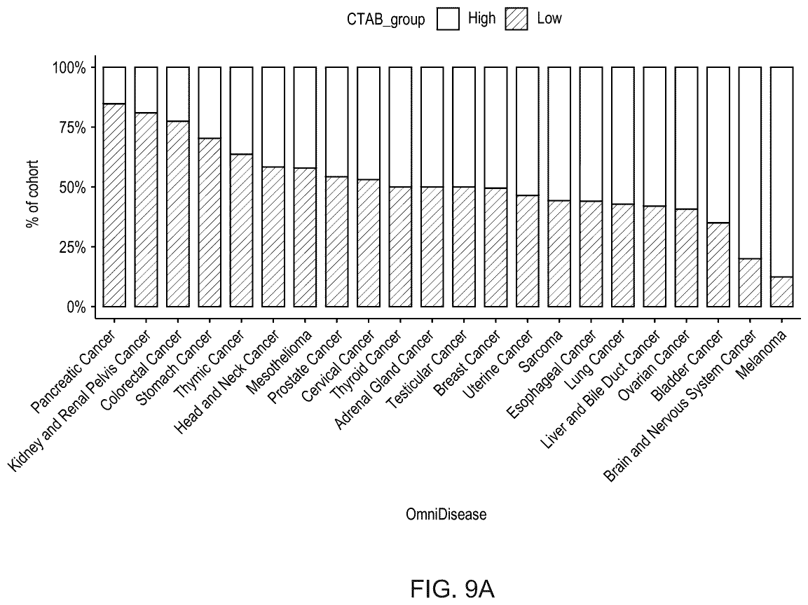

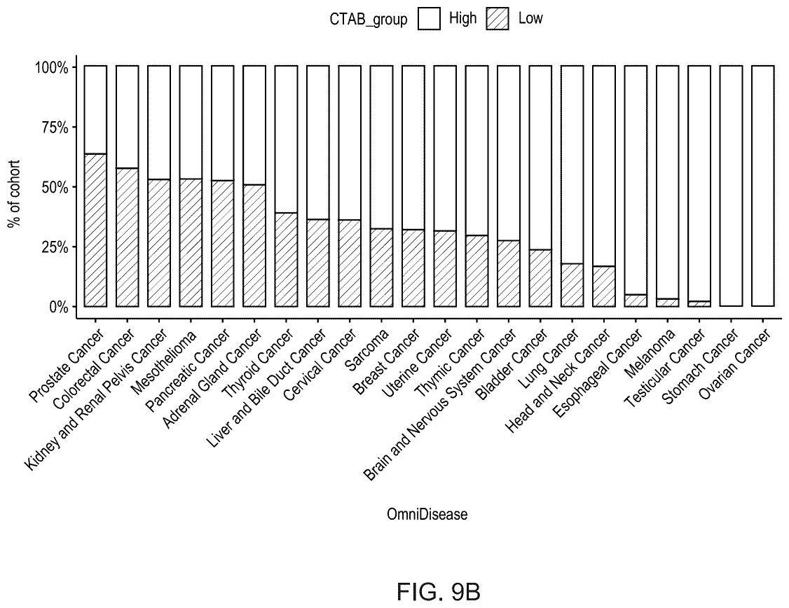

Next, the discovery cohort was divided into CTAB high and CTAB low groups. CTAB scores in 22 distinct types of tumors revealed different mean CTAB among the tumors evaluated (FIG. 9A). The highest CTAB distribution was observed in melanoma and lowest in pancreatic cancer. This suggests that some cancer types, such as melanoma, inherently had higher CTA expression, while other cancer types, such as kidney or colorectal cancer, generally exhibited lower overall CTA expression. Similar observations were made for the independently assembled TCGA cohort (FIG. 9B). This apparent connection between CTA expression and cancer type was particularly relevant in a treatment selection context, where treatments relying on the immunogenicity or targetability of CTA may be more applicable to some cancer types than others. This characteristic CTA expression also suggested that potential modifications or normalizations of CTAB may be possible to maximize its utility in each cancer type. Overall, the CTAB distribution is maintained across the discovery cohort and TCGA cohort, as well as across a wide range of tumor types, supporting CTAB as valid and histology agnostic classifier.

### 5. NSCLC Survival and Treatment Response Analysis

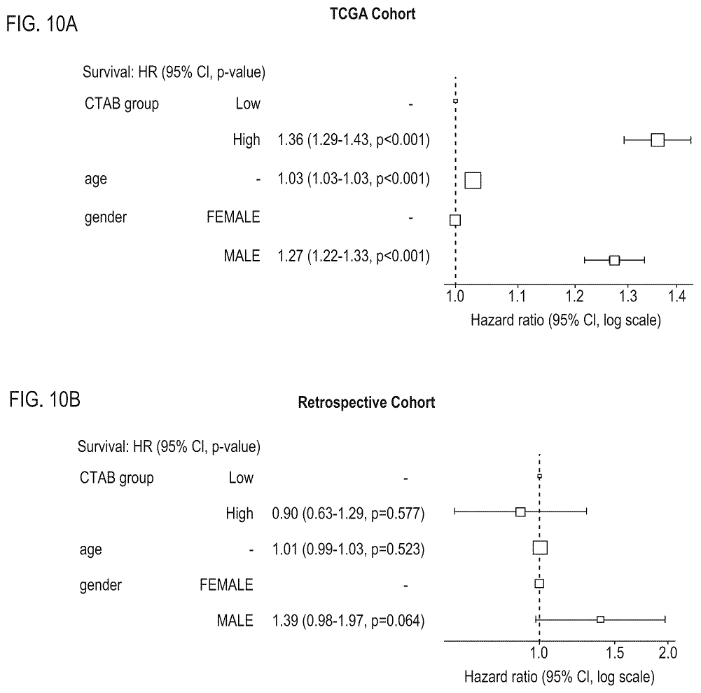

As shown in FIG. 10, overall survival analysis was performed on the TCGA cohort (FIG. 10A) and the retrospective cohort (FIG. 10B) using a CoxPH regression model to determine the Hazard Ratio (HR). The CoxPH regression analysis revealed a significant association between the CTAB threshold classifier and overall survival in the ICB-treated retrospective cohort (HR: 1.36, p<0.001), while no significant association was found in the retrospective cohort (HR=0.90, p=0.577). Notably however, the direction of this association differed between the two cohorts, with high-CTAB samples (e.g., positive CTAB) having better survival in the ICB-treated retrospective cohort and worse survival in the non-ICB-treated TCGA.

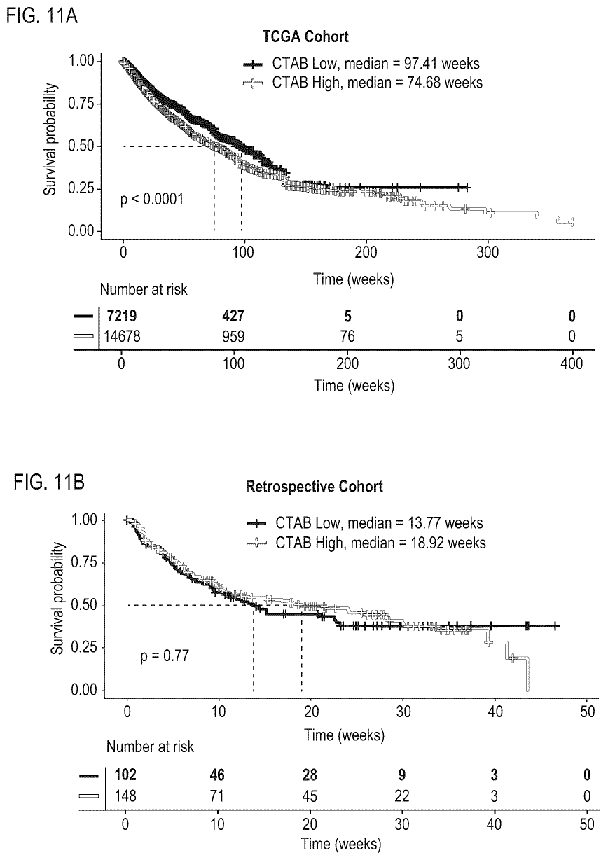

Kaplan-Meier survival analyses comparing high/positive CTAB (≥171) and low/negative CTAB (<171) groups for TCGA (FIG. 11A) and retrospective cohort (FIG. 11B). Kaplan-Meier revealed a strong association (p<0.0001) between positive CTAB status and worse survival in the TCGA, as show in FIG. 11A by the white “+” symbol. This association did not exist in the retrospective cohort (p=0.77), though positive CTAB status (indicated by the black “+” symbol) trended toward better survival (FIG. 11B). This difference suggests that advances in immunotherapy targeting CTA have largely eliminated the survival disbenefit observed in the pre-immunotherapy TCGA. Overall, when evaluating the ICB-treated (e.g., the retrospective cohort) and non-ICB-treated (e.g., the TCGA) cohorts, CTAB demonstrated the ability to predict overall survival, pointing to the utility of ICB in supporting CTA-specific natural immune response.

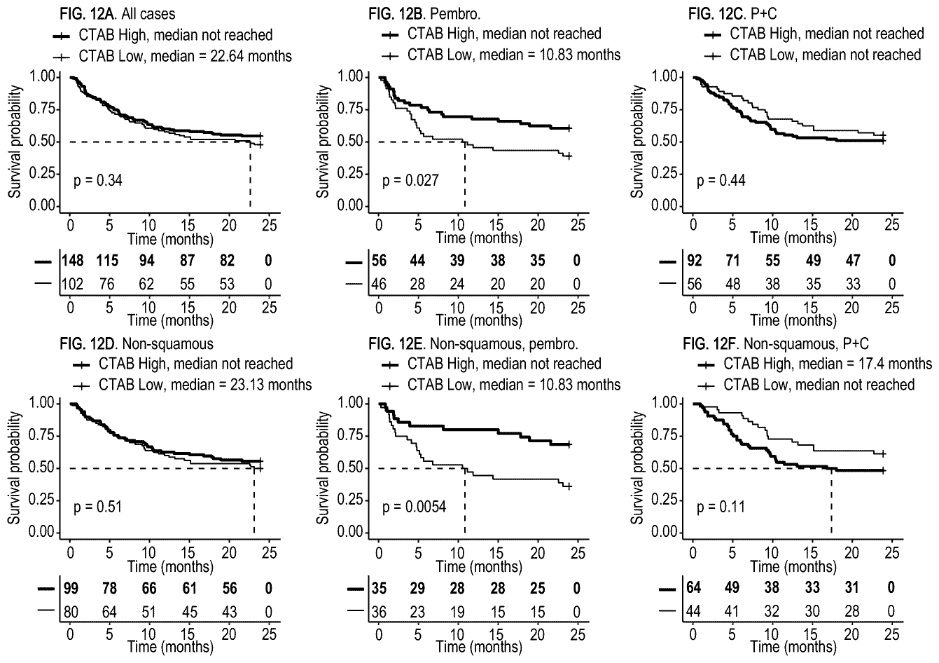

To determine the relationship between CTAB and survival, KM overall survival analyses was performed on the retrospective cohort of 250 subjects with NSCLC treated with anti-PD-1 immunotherapy (pembrolizumab) alone or in combination with chemotherapy (Table 7). The median CTAB score for the discovery cohort of 171 was applied to the NSCLC retrospective cohort as cutoff for CTAB “high” and “low”. Analyses revealed no significant difference in survival between high and low CTAB groups in the overall cohort (FIG. 12A). However, among subjects treated with pembrolizumab monotherapy, the CTAB high group demonstrated significantly better survival [CTAB high: median not reached, CTAB low: median=10.83 months; p=0.027] (FIG. 12B). This trend was not observed among subjects treated with combined pembrolizumab and chemotherapy (FIG. 12C), suggesting that an immunotherapy-only course of treatment may not be best suited for some subjects. This further indicates that the CTAB status of a subject could be an important treatment selection biomarker. To evaluate histologically derived survival differences, the retrospective cohort based was divided based on histology (non-squamous versus squamous). This revealed that the significant association between CTAB status and survival among the pembrolizumab monotherapy group appeared to be largely driven by non-squamous NSCLC (FIGS. 12D-F), which constituted 85% of the retrospective cohort (Table 8). When examined overall, no significant difference in survival between high and low CTAB groups were observed in the non-squamous cohort (FIG. 12D). However, there was a significant relationship when the non-squamous cohort was only treated with pembrolizumab, whereby the CTAB high group had better survival outcomes to the CTAB low group increased [p=0.0054] (FIG. 12E). This trend was not observed among subjects with non-squamous tumors treated with combined pembrolizumab and chemotherapy (FIG. 12F). This suggests that a biological difference between squamous and non-squamous NSCLC subtypes may underlie a greater role played by CTA expression in the immunotherapy-induced immune response.

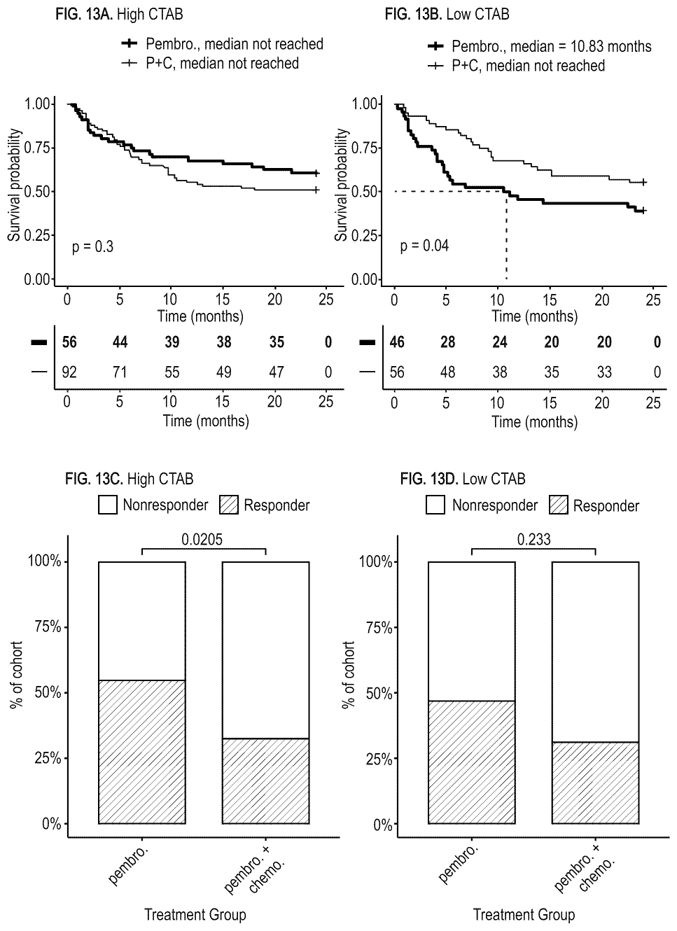

To include outcome metrics other than survival in the evaluation of CTAB as a biomarker, treatment response within the retrospective cohort was also considered. Accordingly, additional KM survival analyses comparing the pembrolizumab monotherapy and combination therapy groups within the CTAB high and CTAB low groups was performed (FIG. 13). Within the CTAB high group, no significant difference in survival outcomes was observed between the two treatment groups (FIG. 13A). However, within the CTAB low group, the combination therapy group exhibited significantly better survival than the pembrolizumab monotherapy group [pembrolizumab monotherapy median=10.83 months, combination therapy: median not reached; p=0.04] (FIG. 13B). Subsequent treatment response rate analysis revealed that among subjects with high CTAB, the pembrolizumab monotherapy group had a significantly greater proportion of subjects responding to treatment than the combination therapy group [p=0.02] (FIG. 13C). When the responder group only included partial or complete responses (PR or CR), a larger proportion of subjects with stable disease (SD) was observed in the combination group as opposed to the monotherapy group [p=0.053] (FIG. 14A). Although the pembrolizumab monotherapy group also showed a higher proportion of responders among low CTAB subjects and a similar distribution of stable disease subjects in both treatment groups (FIG. 14B), the difference was not statistically significant (FIG. 13D). These results suggest that while, among subjects with high CTAB, the addition of chemotherapy to pembrolizumab does not result in a significant survival difference, this change in treatment does affect the nature of the response of these subjects to this drug, perhaps through the suppression of immune cell proliferation within tumors. Taken alongside the survival analyses, these results suggest that in concert with other biomarkers commonly in use for treatment selection such as PD-L1, CTAB may allow for a more nuanced identification of subject subpopulations as candidates or non-candidates for immunotherapy.

### 6. Biomarker Integrations with CTAB

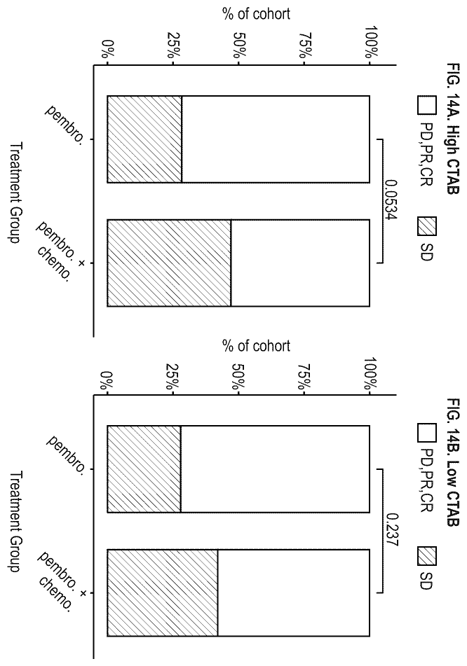

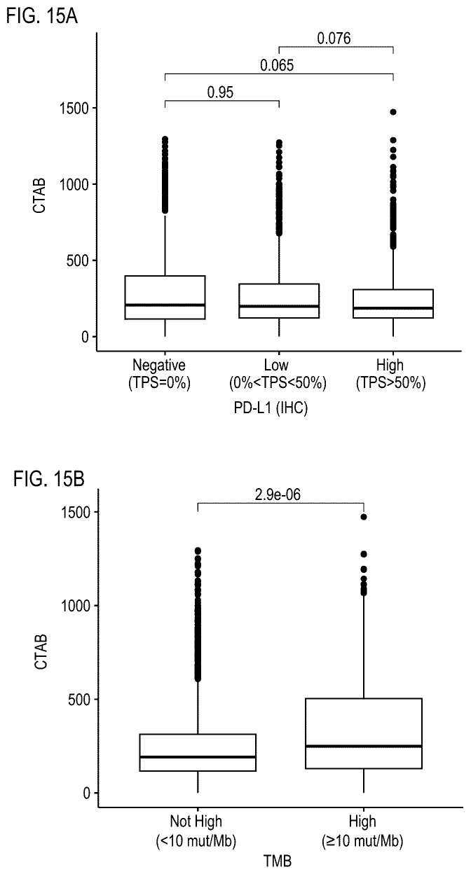

To investigate the statistical relationship between CTAB and previously established biomarkers of response to checkpoint inhibition in solid tumors, the discovery cohort was assessed first by tumor proportion score (TPS), which measure PD-L1 protein expression, and then by tumor mutational burden (TMB). When the discovery cohort was subdivided by PD-L1 classification by IHC, no significant difference in CTAB was observed between the negative (TPS=0%), low (0%<TPS<50%), and high (TPS≥50%) subgroups (FIG. 15A). This suggests that CTAB interrogates aspects of the tumor microenvironment distinct from those assessed by PD-L1 expression alone. When the discovery cohort was similarly subdivided based on TMB, a significant difference was observed between TMB<10 mut/Mb and TMB≥10 mut/Mb subgroups [p=2.9×10⁻⁶] (FIG. 15B). Though CTAB and TMB nominally assess distinct aspects of the tumor microenvironment, the association between these two biomarkers suggests that CTAB, like TMB, is at least partially driven by cell proliferation activity. However, the development of tumor antigens in CTAB differs from TMB. CTA are proteins associated with proliferative activity expressed in cancer cells in tissues that do not normally express them, whereas the neoantigens assessed by TMB are mutated forms of many other genes expressed because of extensive cancer cell proliferation. Additionally, taken together, these results suggest that CTAB assesses microenvironmental features not entirely captured by traditional immune biomarkers like PD-L1 and TMB while still originating from phenomena associated with tumor growth and development.

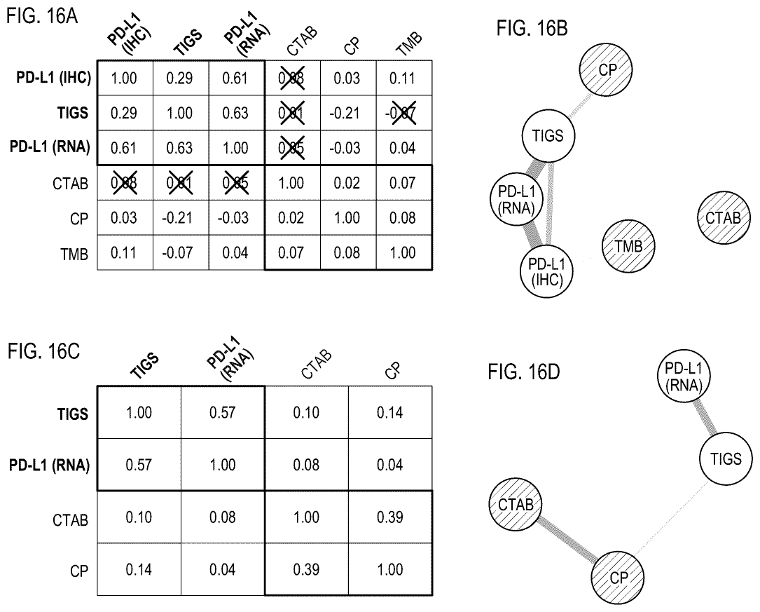

The discovery cohort was further evaluated by CGIP of the tumor immune microenvironment to identify correlations between CTAB and other immune biomarkers. Individual and combination biomarker assessment included PD-L1 IHC (i.e., PD-L1 protein expression), PD-L1 assessed by RNA-seq (i.e. PD-L1 gene expression), TMB, tumor immunogenic signature (TIGS), cell proliferation (CP) and CTAB (FIGS. 16A and 16B). These comparisons were also assed in the TCGA cohort (FIGS. 16C and 16D). These analyses revealed weak or nonsignificant correlations between CTAB and these other biomarkers, indicating that CTAB measures aspects of the tumor microenvironment independent from these biomarkers.

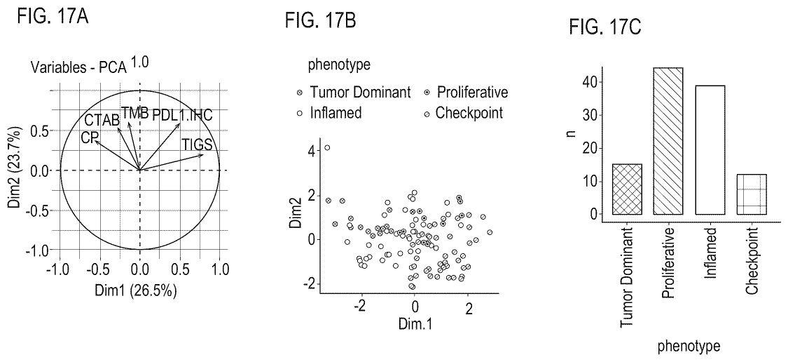

As shown in FIG. 17, analysis of the discovery cohort revealed four distinct biomarker combination groups that describe underlying tumor immunobiology phenotypes: tumor dominant (CTAB, TMB, CP High), proliferative (CP High), inflamed (TIGS High), and checkpoint (PDL1, TIGS and TMB High).

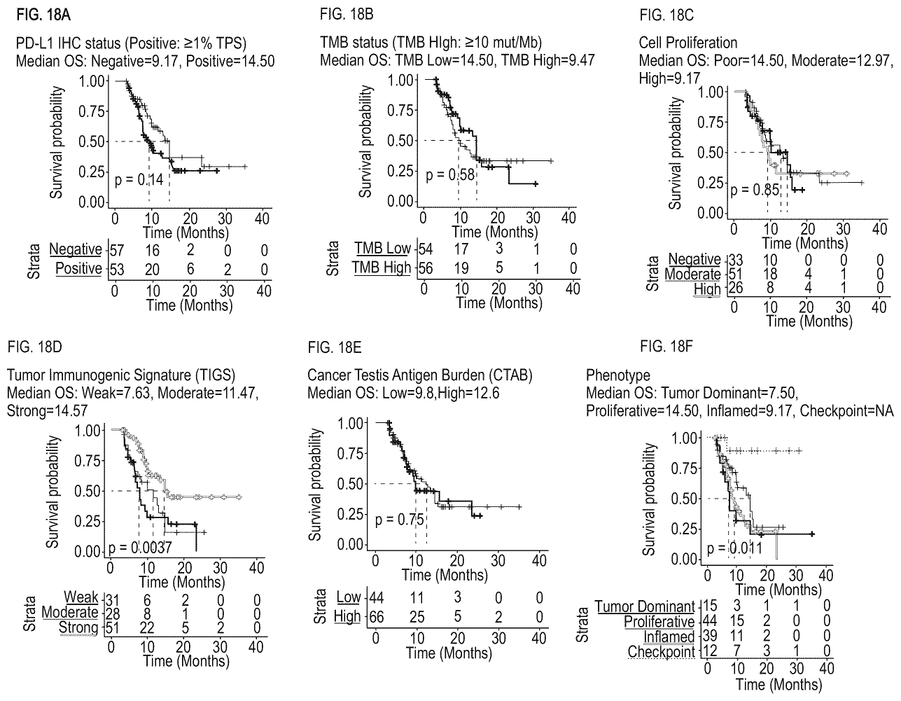

Application of these biomarker groups to a validation cohort of 110 immunotherapy-treated NSCLC patients (Table 10) demonstrated significant differences in ICI outcomes between groups (FIG. 18). Significant associations with overall survival, assessed by Kaplan-Meier survival analysis, were not observed in this cohort for groupings based on PD-L1 IHC (FIG. 18A), TMB (FIG. 18B), a cell proliferation gene expression signature (FIG. 18C), or cancer testis antigen burden (FIG. 18E) alone. However, tumor inflammation, as assessed by the TIGS gene expression signature, was found to be associated with overall survival (p=0.0037, FIG. 18D). Notably, the four phenotype groups developed by combining these biomarkers were found to be significantly associated with overall survival in this validation cohort (p=0.011, FIG. 18F). Importantly, this phenotype stratification demonstrated greater increase in median survival than stratification by any constituent biomarker (FIG. 18F).

TABLE 10
ICI-treated validation cohort composition.

| Variable | Group | N (% of cohort) | |
|---|---|---|---|
| Age at initial diagnosis | <30 | 1 | (0.9%) |
| (years) | 30-39 | 0 | (0.0%) |
| | 40-49 | 3 | (2.7%) |
| | 50-59 | 26 | (23.6%) |
| | 60-69 | 41 | (37.3%) |
| | 70-79 | 30 | (27.3%) |
| | ≥80 | 9 | (8.2%) |
| | Mean | 65.4 | |
| Sex | Female | 58 | (52.7%) |
| | Male | 52 | (47.3%) |
| Race | White | 91 | (82.7%) |
| | Other | 14 | (12.7%) |
| | No Data | 5 | (4.5%) |
| Vital status at last | Alive | 55.00 | (50.0%) |
| follow up | Dead | 55.00 | (50.0%) |
| Checkpoint inhibitor | atezolizumab | 2 | (1.8%) |
| | ipilimumab + nivolumab | 2 | (1.8%) |
| | nivolumab | 71 | (64.5%) |
| | pembrolizumab | 35 | (31.8%) |
| Months of follow up | <1 | 48 | (43.6%) |
| | 3 | 6 | (5.5%) |
| | 6 | 17 | (15.5%) |
| | 10 | 22 | (20.0%) |
| | >10 | 17 | (15.5%) |

| | | |
|---|---|---|
| | Median | 8 |
| Year of diagnosis | Range | 2007-2017 |

| | | |
|---|---|---|
| All Samples | 110 | (100%) |

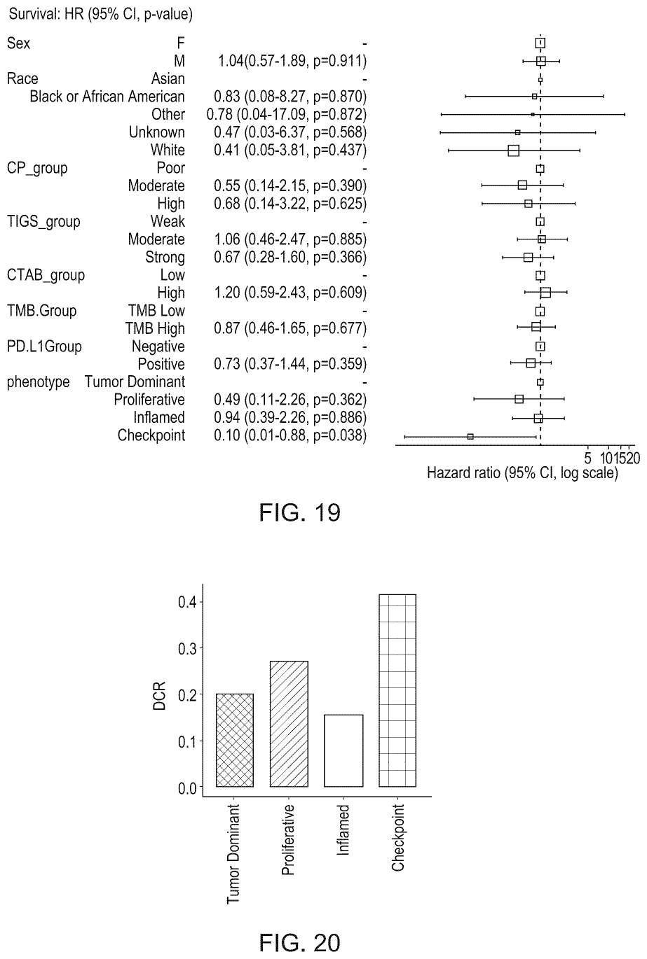

CoxPH survival analysis was performed using sex, race, the five constituent biomarkers, and phenotype as survival predictors (FIG. 19). Out of all the predictors, only the checkpoint tumor phenotype displayed a significant decreased in hazard ratio [HR=0.10; p=0.038] for ICI treatment. This indicates that the checkpoint group constitutes as a survival predictor biomarker.

Finally, the disease control rate (DCR) for the 110 NSCLC subject validation cohort was assessed (FIG. 20). A significant association with ICI response was found by classifying the retrospective cohort into the four groups, with the following results: tumor dominant [DCR=0.200]; proliferative [DCR=0.273], inflamed [DCR=0.154]; and checkpoint [DCR=0.417]. The checkpoint group was overrepresented by the highest proportion of disease control [p=0.0313].

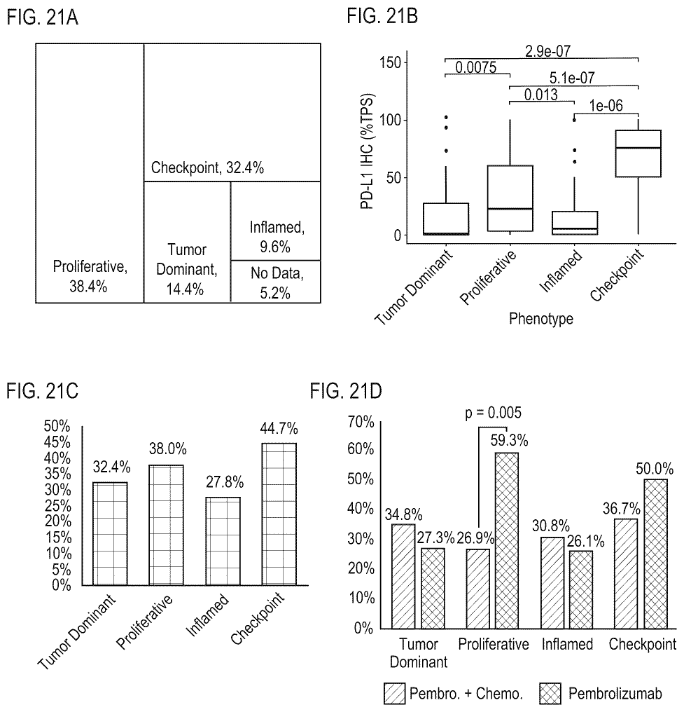

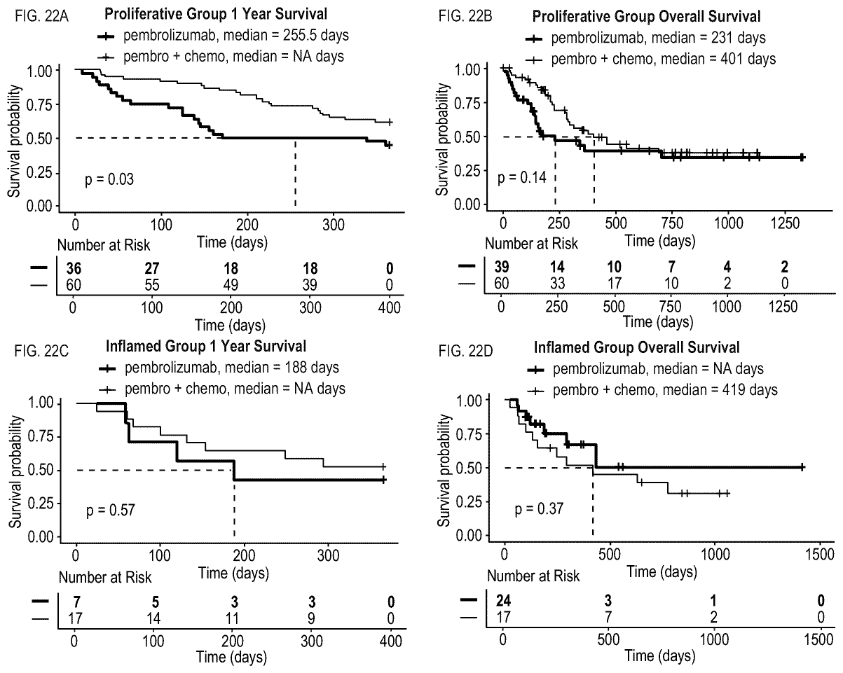

In order to compare the survival and response implications of this phenotype classification between patients treated with combination immunotherapy and chemotherapy and those treated with immunotherapy alone, the same phenotype analysis was applied to the 250-patient retrospective cohort (Table 8). Within this cohort, the largest proportion of tumor samples were found to be of a proliferative phenotype (38.4%), followed by the checkpoint (32.4%), tumor dominant (14.4%), and inflamed (5.2%) phenotypes (FIG. 21A). These phenotype groups were found to have significant differences in PD-L1 expression (as assessed by IHC), with only the proliferative and inflamed phenotypes not showing a significant difference in PD-L1 expression from one another (FIG. 21B). However, no significant difference in ORR between these phenotype groups was observed (FIG. 21C). To assess both the effect of multiple treatment regiments in each phenotype group and the effects of phenotype on the response to different treatments, ORR, OS, and 1 year survival were compared between the pembrolizumab and pembrolizumab-chemotherapy combination groups within each phenotype (FIG. 21D, FIGS. 22A-22D). Subjects in the proliferative group (median PD-L1=20% TPS) treated with single agent pembrolizumab showed a significantly higher ORR (59.3% vs. 26.9%; p-0.005; 21D), significantly improved 1-year overall survival (p=0.03; FIG. 22A) and trended toward better overall survival (p=0.14; FIG. 22B) compared to those treated with pembrolizumab+chemo. Importantly, among subjects in the inflamed group (median PD-L1=1% TPS), those treated with pembrolizumab+chemo did not demonstrate significantly different ORR (FIG. 21D), 1-year overall survival (FIG. 22C) or OS (FIG. 22D) compared to those treated with single agent pembrolizumab. These results suggest that PD-L1 low NSCLC subjects with a proliferative phenotype may derive greater benefit from single agent pembrolizumab than from pembrolizumab+chemo, whereas PD-L1 low subjects with an inflamed phenotype appear to derive similar benefit from either treatment regimen.

### B. Conclusion

An integrated approach combining comprehensive tumor profiling and emerging biomarkers better predicts ICI response and survival in multiple histologies. Divergent outcomes between the resulting groups are likely the result of distinct tumor-immune interaction modalities. Furthermore, a novel pan-cancer biomarker of tumor immune microenvironment measuring CTA co-expression in solid tumors was developed. This biomarker aids in deepening the understanding of response to cancer treatments such as immunotherapies, ICIs, cell therapies and cancer vaccines. A comprehensive genomic and immune profiling strategy benefit treatment selection as well clinical trial strategies that mitigate multitude of immune escape mechanisms driving the tumor growth. The results show that in combination with other orthogonal biomarkers assessing distinct aspects of the tumor-immune microenvironment, CTAB is an important part of a complete understanding of the tumor microenvironment and its implications for treatment.

### Materials and Methods

### 1. Quality Assessment of Clinical Formalin Fixed Paraffin Embedded Tissue Specimens

Tissue sections from formalin fixed paraffin embedded (FFPE) blocks were cut to a thickness of 5 μm onto positively charged slides. The tissue sections comprised tumor samples from the discovery cohort (N=5,250 samples representing 22 tumor types) and the retrospective cohort (N=250 immunotherapy-treated NSCLC). Of the 250 NSCLC tumors in the retrospective cohort, 103 (41%) of those tumors were treated with pembrolizumab monotherapy and the remaining 147 (59%) were treated with a combination of pembrolizumab and chemotherapy. One section from each tissue sample was stained with hematoxylin and eosin and examined by a board-certified anatomical pathologist to assess the adequacy of tumor representation, presence of necrosis or issues with fixation or handling, and quality of tissue preservation. Specimens with less than 5% tumor tissue and more than 50% necrosis were excluded from analysis. To achieve the assay requirements for RNA (10 ng) and DNA (20 ng) input, tissue from 3-5 unstained slide sections was required, with or without tumor microdissection.

### 2. Nucleic Acid Isolation

DNA and RNA were co-extracted from each tissue sample and processed for gene expression analysis by RNA-seq and TMB analysis by DNA-seq. The extracted nucleic acids were then quantified using a Qubit fluorometer (ThermoFisher Scientific), which uses ribogreen staining for RNA and picogreen staining for DNA. PD-L1 status of each tumor was assessed by IHC.

### 3. Genomic and Immune Profiling

Cohorts were tested by comprehensive genomic and immune profiling (CGIP), including tumor mutational burden (TMB) and the mRNA expression of 17 CTAs. Gene expression was evaluated by RNA sequencing for 395 transcripts on samples that met validated quality control thresholds. Potential pre-analytical interference to the gene expression values was assessed during assay validation. TMB was measured by DNA sequencing of the full coding region of 409 cancer related genes as non-synonymous mutations per megabase (mut/Mb) of sequenced DNA on samples with >30% tumor nuclei and TSO500 genomic profiling. To perform the RNA-seq and DNA-seq analyses, libraries of the extracted nucleic acids were prepared and sequenced to appropriate depth on the Ion Torrent S5XL sequencer (ThermoFisher Scientific) and NovaSeq 6000 (Illumina). PD-L1 expression tumor proportion score (TPS) was assessed by IHC (22C3).

### 4. Data Analysis

The RNA-seq data was processed using the Torrent Suite plugin immuneResponseRNA (ThermoFisher Scientific), which generated absolute reads for each transcript. For each of the 17 CTA genes, the expression values were then converted to a percentile rank of 0-100 when compared to a reference population of 735 solid tumors of 35 histologies. The CTAB biomarker was calculated by summing the gene expression ranks of 17 CTAs (BAGE, CTAG1B (NY-ESO-1), CTAG2 (LAGE-1A), GAGE1, GAGE10, GAGE12J, GAGE13, GAGE2, MAGEA1, MAGEA10, MAGEA12, MAGEA3, MAGEA4, MAGEC2, MLANA, SSX2, and XAGE1B), resulting in an integer value between 0 and 1700 for each sample with gene expression data for all 17 CTAs. The median CTAB score for the discovery cohort of 171 was applied to the NSCLC retrospective cohort as cutoff for CTAB “high” and “low”. Genomic profiling was performed using Illumina TSO500 analysis pipeline [Illumina: v2.1.0.60]. All subsequent data analyses were performed using R (v4.3.0). To analyze the relationship between CTA gene expression and clinical outcomes, Spearman correlation (ρ or rₛ) analysis was performed. Network graph visualization of the correlations between CTAs was performed using a Fruchterman-Reingold force-directed algorithm to arrange nodes representing each CTA connected by edges weighted according to the absolute value of the Spearman correlation observed between each pair of CTAs. Continuous variables were compared between subject groups using Kruskal-Wallis or Wilcoxon Rank-Sum tests as appropriate. Kaplan-Meier survival analyses were used to detect overall survival (OS) differences between subgroups in the NSCLC cohort using two-year survival data. Objective response rate (ORR) based on RECIST criteria (e.g., treatment response) was compared between subgroups using Fisher's exact test without continuity correction. In addition, two previously published gene expression signatures were calculated: the cell proliferation (CP) signature and the tumor immunogenic signature (TIGS), which measures the “hot” or “cold” inflammation state of the tumor microenvironment. P-values less than 0.05 were considered significant.

### VI. ADDITIONAL CONSIDERATIONS

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium”, “storage” or “memory” can represent one or more memories for storing data, including read only memory (ROM), random access memory (RAM), magnetic RAM, core memory, magnetic disk storage mediums, optical storage mediums, flash memory devices and/or other machine-readable mediums for storing information. The term “machine-readable medium” includes but is not limited to portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

What is claimed is:

1. A method of treating a subject having cancer comprising:
obtaining sequencing reads from a biological sample from the subject, wherein the sequencing reads are generated from a sequencing assay,
measuring, using the sequencing reads, expression of one or more cancer testis antigen (CTA) genes in the biological sample from the subject having cancer,
determining a cancer testis antigen burden (CTAB) score based on the measured expression of the one or more genes,
classifying the CTAB score as a high-CTAB score or a low-CTAB score compared to a reference CTAB score,
in response to the CTAB score being a high-CTAB score, treating the subject with a treatment; and
in response to the CTAB score being a low-CTAB score, treating the subject with a different treatment.

2. The method of claim 1, wherein the biological sample comprises cells, tissue, or biological fluid.

3. The method of claim 2, wherein the biological sample is a tissue.

4. The method of claim 1, wherein the treatment is an immune checkpoint inhibitor, or wherein the treatment is a combination of treatments comprising an immune checkpoint inhibitor and an additional anti-cancer therapy.

5. The method of claim 4, wherein the immune checkpoint inhibitor is pembrolizumab, ipilimumab, nivolumab, atezolizumab, cemiplimab, avelumab, or durvalumab.

6. The method of claim 5, wherein the immune checkpoint inhibitor is pembrolizumab.

7. The method of claim 4, wherein the additional anti-cancer therapy comprises surgery, chemotherapy, chemoradiation, anticancer drugs, anti-VEGF therapy, novel cancer treatments, or any combination thereof.

8. The method of claim 7, wherein the additional anti-cancer therapy is chemotherapy.

9. The method of claim 1, wherein the different treatment is an immune checkpoint inhibitor, or wherein the different treatment is a combination of treatments comprising an immune checkpoint inhibitor and an additional anti-cancer therapy.

10. The method of claim 9, wherein the immune checkpoint inhibitor is pembrolizumab, ipilimumab, nivolumab, atezolizumab, cemiplimab, avelumab, or durvalumab.

11. The method of claim 9, wherein the additional anti-cancer therapy comprises surgery, chemotherapy, chemoradiation, anticancer drugs, anti-VEGF therapy, novel cancer treatments, or any combination thereof.

12. The method of claim 11, wherein the additional anti-cancer therapy is chemotherapy.

13. The method of claim 1 further comprising repeating the treatment at a later timepoint to determine the efficacy of the treatment.

14. The method of claim 1 further comprising:
obtaining slides of the biological sample, wherein:
the biological sample is a tumor tissue sample, and
at least one slide is stained using hematoxylin and eosin staining, and the remaining slides are unstained;
examining the at least one stained slide for pathological features associated with cancer to determine the severity of the cancer.

15. The method of claim 14, wherein the pathological features comprise nuclear atypia, mitotic activity, tumor necrosis, different patterns of invasion, tumor stroma, non-invasive urothelial carcinoma, invasive urothelial carcinoma, low-grade tumors, high-grade tumors, squamous cell differentiation, glandular differentiation, or any combination thereof.

16. A method for identifying a response to a treatment in a subject having cancer comprising:
(a) obtaining slides of a biological sample from a subject, wherein at least one slide is stained, and the remaining slides are unstained;
(b) examining the at least one stained slide for pathological features associated with cancer to determine whether the subject is cancer positive, indeterminant, or cancer negative;
(c) in response to the subject being cancer positive or indeterminant:
(i) obtaining sequencing reads from the remaining unstained slides, wherein the sequencing reads are generated from a sequencing assay,
(ii) measuring, using the sequencing reads, expression of one or more cancer testis antigen (CTA) genes,
(iii) determining a cancer testis antigen burden (CTAB) score based on the measured expression of the one or more genes,
(iv) classifying the CTAB score as a high-CTAB score or a low-CTAB score compared to a reference CTAB score, and
(v) identifying the subject as responsive to the treatment, wherein (i) the high-CTAB score is indicative of responsiveness to the treatment, and (ii) the low-CTAB score is not indicative of responsiveness to the treatment; and
(d) in response to the subject being cancer negative: not performing steps (i)-(v).

17. The method of claim 16, wherein the treatment is an immune checkpoint inhibitor, or wherein the treatment is a combination of treatments comprising an immune checkpoint inhibitor and an additional anti-cancer therapy.

18. The method of claim 17, wherein the immune checkpoint inhibitor is pembrolizumab, ipilimumab, nivolumab, atezolizumab, cemiplimab, avelumab, or durvalumab.

19. The method of claim 18, wherein the immune checkpoint inhibitor is pembrolizumab.

20. The method of claim 17, wherein the additional anti-cancer therapy comprises surgery, chemotherapy, chemoradiation, anticancer drugs, anti-VEGF therapy, novel cancer treatments, or any combination thereof.

21. The method of claim 20, wherein the additional anti-cancer therapy is chemotherapy.

22. The method of claim 16, wherein the biological sample comprises cells, tissue, or biological fluid.

23. The method of claim 22, wherein the biological sample is a tissue.

24. The method of claim 22, wherein the biological sample is collected from a tissue suspected of being cancerous.

25. The method of claim 16, wherein the at least one stained slide is stained using histological staining techniques.

26. The method of claim 25, wherein the histological staining technique is hematoxylin and eosin staining.

27. The method of claim 16, wherein the pathological features comprise nuclear atypia, mitotic activity, tumor necrosis, different patterns of invasion, tumor stroma, non-invasive urothelial carcinoma, invasive urothelial carcinoma, low-grade tumors, high-grade tumors, squamous cell differentiation, glandular differentiation, or any combination thereof.

28. The method of claim 16, wherein the subject having cancer has pancreatic, kidney, renal, pelvic, colorectal, stomach, thymic, head and neck, mesothelial, prostate, cervical, thyroid, adrenal, testicular, breast, uterine, bone, esophageal, lung, liver, bile duct, ovarian, bladder, nervous system, or skin cancer.

29. The method of claim 28, wherein the cancer is lung cancer.

30. The method of claim 29, wherein the lung cancer is non-small cell lung cancer (NSCLC).

31. The method of claim 16, wherein the sequencing assay is RNA-sequencing, DNA-sequencing, or both.

32. The method of claim 16, wherein the one or more genes are a set of genes selected from the genes identified in Tables 1-5.

33. The method of claim 16, wherein the CTAB score is determined by measuring the expression of each of the one or more genes for the CTAs as a percentile rank at a gene level and summing the percentile ranks at a sample level to derive the CTAB score.

34. The method of claim 16, wherein the CTAB score represents aspects of the tumor microenvironment in the biological sample that are not measured by current standard of care testing.

35. The method of claim 34, the current standard of care testing comprises PD-L1 protein expression, PD-L1 gene expression, tumor mutational burden, tumor immunogenic signature, cell proliferation, or any combination thereof.

36. The method of claim 16, wherein the high CTAB score is a CTAB score≥the reference CTAB score and a low-CTAB score is CTAB score<the reference CTAB score.

37. The method of claim 36, wherein the reference CTAB score is calculated as a mean or median CTAB score from a reference population having a same tumor or cancer type as the subject.

38. The method of claim 37, wherein calculating the reference CTAB score comprises determining the CTAB score for all subjects within the reference population, calculating the mean or median for the CTAB scores determined for all the subjects, and assigning the mean or median as the reference CTAB score for the given tumor or cancer type.

39. The method of claim 16 further comprising when the subject has the high CTAB score, administering an effective amount of the treatment, and wherein when the subject has the low CTAB score administering an effective amount of a different treatment.

40. The method of claim 39, wherein the different treatment is a combination of pembrolizumab and chemotherapy.

41. A method of identifying a subject having NSCLC cancer who will benefit from a treatment comprising an immunotherapy, the method comprising:
determining a cancer testis antigen burden (CTAB) score from a sample from the individual, wherein:
the CTAB score is determined by measuring expression of each of one or more genes for cancer testis antigens (CTAs) as a percentile rank at a gene level and summing the percentile ranks at a sample level to derive the CTAB score, and
the one or more genes comprise MAGEA10, MAGEA4, GAGE12J, GAGE2, GAGE1, GAGE13, SSX2, CTAG1B, CTAG2, BAGE, MAGEC2, MAGEA1, MAGEA12, MAGEA3, MLANA, XAGE1B, GAGE10, or any combination thereof; and
identifying the subject as one who will respond to the treatment comprising immunotherapy when the CTAB score is at or above a reference CTAB score.

42. The method of claim 41, in response to as one who will benefit from the treatment comprising immunotherapy, treating the subject with an effective amount of the immunotherapy.

43. The method of claim 42, wherein the immunotherapy is an immune checkpoint inhibitor including pembrolizumab, ipilimumab, nivolumab, atezolizumab, cemiplimab, avelumab, or durvalumab.

44. The method of claim 43, wherein the immunotherapy is pembrolizumab.

45. The method of claim 41, wherein the reference CTAB score is calculated as a mean or median CTAB score from a reference population having a same tumor or cancer type as the subject.

46. The method of claim 41, further comprising identifying the subject as one who is not responsive to the treatment comprising immunotherapy when the CTAB score is below the reference CTAB score.

47. A method of selecting a treatment for a subject having NSCLC cancer, the method comprising:
determining a cancer testis antigen burden (CTAB) score from a sample from the individual, wherein:
the CTAB score is determined by measuring expression of each of one or more genes for cancer testis antigens (CTAs) as a percentile rank at a gene level and summing the percentile ranks at a sample level to derive the CTAB score, and
the one or more genes comprise MAGEA10, MAGEA4, GAGE12J, GAGE2, GAGE1, GAGE13, SSX2, CTAG1B, CTAG2, BAGE, MAGEC2, MAGEA1, MAGEA12, MAGEA3, MLANA, XAGE1B, GAGE10, or any combination thereof;
identifying the subject as one who will benefit from a treatment when the CTAB score is at or above a reference CTAB score; and
in response to identifying the subject as one who will benefit from the treatment, selecting immunotherapy alone or in combination with an additional anti-cancer therapy other than the immunotherapy for the subject based on the CTAB score.

48. The method of claim 47, in response to the selecting, treating the subject with the immunotherapy alone or in combination with the additional anti-cancer therapy other than the immunotherapy.

49. The method of claim 47, wherein the immunotherapy is an immune checkpoint inhibitor including pembrolizumab, ipilimumab, nivolumab, atezolizumab, cemiplimab, avelumab, or durvalumab.

50. The method of claim 49, wherein the immunotherapy is pembrolizumab.

51. The method of claim 48, wherein the additional anti-cancer therapy comprises surgery, chemotherapy, chemoradiation, anticancer drugs, anti-VEGF therapy, novel cancer treatments, or any combination thereof.

52. The method of claim 51, wherein the additional anti-cancer therapy is chemotherapy.

53. The method of claim 47, wherein the reference CTAB score is calculated as a mean or median CTAB score from a reference population having a same tumor or cancer type as the subject.

54. The method of claim 47, further comprising identifying the subject as one who is less likely to benefit from the treatment comprising immunotherapy when the CTAB score is below the reference CTAB score, and in response to identifying the subject as one who is less likely to benefit from the treatment, selecting an anti-cancer therapy for the subject other than immunotherapy based on the CTAB score.

55. An assay system, comprising:
an assay surface comprising a chip, array, fluidity card, micro-well plate, or a combination thereof; and
(i) nucleic acid probes that comprise complementary nucleic acid sequences to at least 10 to 50 nucleic acid sequences of a set of genes consisting of: MAGEA10, MAGEA4, GAGE12J, GAGE2, GAGE1, GAGE13, SSX2, CTAG1B, CTAG2, BAGE, MAGEC2, MAGEA1, MAGEA12, MAGEA3, MLANA, XAGE1B, and GAGE10, (ii) antibodies or antigen-binding fragments of antibodies that target polypeptides encoded by the set of genes consisting of: MAGEA10, MAGEA4, GAGE12J, GAGE2, GAGE1, GAGE13, SSX2, CTAG1B, CTAG2, BAGE, MAGEC2, MAGEA1, MAGEA12, MAGEA3, MLANA, XAGE1B, and GAGE10, or (iii) both.

56. A kit comprising a plurality of nucleic acids, wherein the plurality of nucleic acids are at least 5 nucleotides in length and are at least 95% identical to a 5 nucleotide continuous sequence or are at least 95% identical to a sequence complementary to the 5 nucleotide continuous sequence within at least two genes selected from the group consisting of MAGEA10, MAGEA4, GAGE12J, GAGE2, GAGE1, GAGE13, SSX2, CTAG1B, CTAG2, BAGE, MAGEC2, MAGEA1, MAGEA12, MAGEA3, MLANA, XAGE1B, and GAGE10.
